(19) United States
(12) Patent Application Publication
(10) Pub. No.: US 2025/0241705 A1
(43) Pub. Date: Jul. 31, 2025

(54) CATHETER ASSEMBLIES AND SYSTEMS WITH FLEXIBLE WORKING SITE SECTIONS AND METHODS FOR FORMING FISTULAS

(71) Applicant: TVA MEDICAL GROUP, INC., Franklin Lakes, NJ (US)

(72) Inventors: Oladipo Peter Akerle-ale, Franklin Lakes, NJ (US); Andrew Moll, Franklin Lakes, NJ (US); Alex Palmer, Franklin Lakes, NJ (US); Olivia R. Palmer, Franklin Lakes, NJ (US); Breanna Simpson, Franklin Lakes, NJ (US)

(21) Appl. No.: 18/856,777

(22) PCT Filed: Apr. 13, 2022
(86) PCT No.: PCT/US2022/024593

§ 371 Date: Oct. 14, 2024

(51) Int. Cl. A61B 18/14; A61B 18/00

(52) U.S. Cl. CPC A61B 18/1492; A61B 2018/00404; A61B 2018/00577; A61B 2018/00625

(57) ABSTRACT

A catheter assembly, including a delivery catheter including a delivery catheter body defining an inner lumen of the delivery catheter. The catheter assembly includes a working catheter including a modification device. The working catheter extends through the inner lumen of the delivery catheter, and the working catheter is selectively couplable with the delivery catheter.

### TECHNICAL FIELD

The present disclosure relates to assemblies, systems, and methods for forming a fistula, and more particularly assemblies, systems, and methods with flexible working site sections for increasing contact between the working site section and a vessel for fistula formation.

### BACKGROUND

A fistula is generally a passageway formed between two internal organs. Forming a fistula between two blood vessels can have one or more beneficial functions. For example, the formation of a fistula between an artery and a vein may provide access to the vasculature for hemodialysis patients. Specifically, forming a fistula between an artery and a vein allows blood to flow quickly between the vessels while bypassing the capillaries. In other instances, a fistula may be formed between two veins to form a veno-venous fistula. Generally, fistula formation requires surgical dissection of a target vein, and transecting and moving the vein for surgical anastomosis to the artery. It may therefore be useful to find less invasive and reliable devices and methods for forming a fistula between two blood vessels.

### SUMMARY

One challenging aspect of forming a fistula between blood vessels, though other body vessels are contemplated and possible, is properly aligning and coapting catheters in adjacent blood vessels prior to fistula formation. Accordingly, a need exists for alternative systems, methods, and catheters for fistula formation that ensure catheter alignment and coaptation. Embodiments of the present disclosure are directed to improvements over the above limitations by providing catheter assemblies including a delivery catheter and a flexible working catheter selectively coupled with the delivery catheter.

In one embodiment, a catheter assembly includes a delivery catheter including a delivery catheter body defining an inner lumen of the delivery catheter, and a working catheter including a modification device. The working catheter extends through the inner lumen of the delivery catheter, and the working catheter is selectively couplable with the delivery catheter.

In another embodiment, a method of forming a fistula between a first blood vessel and a second blood vessel, including advancing a first catheter assembly into the first blood vessel. The first catheter assembly includes a delivery catheter including a delivery catheter body defining an inner lumen of the delivery catheter and a working catheter including a modification device. The working catheter extends through the inner lumen of the delivery catheter, and the working catheter is selectively couplable with the delivery catheter. The method further includes decoupling the working catheter from the delivery catheter such that the working catheter and delivery catheter are independently moveable relative to each other and modifying tissue with the modification device of the working catheter.

In another embodiment, a system for forming a fistula between two blood vessels, including a first catheter assembly. The first catheter assembly includes a delivery catheter including a delivery catheter body defining an inner lumen of the delivery catheter and a working catheter including a modification device. The working catheter extends through the inner lumen of the delivery catheter, and the working catheter is selectively couplable with the delivery catheter. The system further includes a second catheter.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a side view of a catheter assembly with a delivery catheter and working catheter of the catheter assembly coupled, according to one or more embodiments shown and described herein;

FIG. 1B schematically depicts a sectional view of the catheter assembly of FIG. 1A, according to one or more embodiments shown and described herein;

FIG. 1C schematically depicts a sectional view of the catheter assembly of FIGS. 1A and 1B with the delivery catheter and the working catheter decoupled, according to one or more embodiments shown and described herein;

FIG. 1D schematically depicts a side view of a sleeve distally advanced over the catheter of FIG. 1A, according to one or more embodiments shown and described herein;

FIG. 2A schematically depicts a side view of another catheter assembly with a delivery catheter and working catheter of the catheter assembly coupled, according to one or more embodiments shown and described herein;

FIG. 2B schematically depicts a sectional view of the catheter assembly of FIG. 2A, according to one or more embodiments shown and described herein;

FIG. 2C schematically depicts a sectional view of the catheter assembly of FIGS. 2A and 2B with the delivery catheter and the working catheter decoupled, according to one or more embodiments shown and described herein;

FIG. 2D schematically depicts a sectional view of the catheter assembly of FIG. 2A with the delivery catheter and the working catheter decoupled, according to one or more embodiments shown and described herein;

FIG. 3A schematically depicts a side view of another catheter assembly with a delivery catheter and working catheter of the catheter assembly coupled, according to one or more embodiments shown and described herein;

FIG. 3B schematically depicts a sectional view of the catheter assembly of FIG. 3A, according to one or more embodiments shown and described herein;

FIG. 3C schematically depicts a sectional view of the catheter assembly of FIGS. 3A and 3B with the delivery catheter and the working catheter decoupled, according to one or more embodiments shown and described herein;

FIG. 3D schematically depicts a sectional view of the catheter assembly of FIG. 3A with the delivery catheter and the working catheter decoupled, according to one or more embodiments shown and described herein;

FIG. 4A schematically depicts a side view of another catheter assembly with a delivery catheter and working catheter of the catheter assembly coupled, according to one or more embodiments shown and described herein;

FIG. 4B schematically depicts a sectional view of the catheter assembly of FIG. 4A, according to one or more embodiments shown and described herein;

FIG. 4C schematically depicts a sectional view of the catheter assembly of FIGS. 4A and 4B with the delivery catheter and the working catheter decoupled, according to one or more embodiments shown and described herein;

FIG. 5A schematically depicts a side view of another catheter assembly with a delivery catheter and working catheter of the catheter assembly coupled, according to one or more embodiments shown and described herein;

FIG. 5B schematically depicts a sectional view of the catheter assembly of FIG. 5A, according to one or more embodiments shown and described herein;

FIG. 5C schematically depicts a sectional view of the catheter assembly of FIGS. 5A and 5B with the delivery catheter and the working catheter decoupled, according to one or more embodiments shown and described herein;

FIG. 6A schematically depicts a side view of another catheter assembly with a delivery catheter and working catheter of the catheter assembly coupled, according to one or more embodiments shown and described herein;

FIG. 6B schematically depicts a sectional view of the catheter assembly of FIG. 6A, according to one or more embodiments shown and described herein;

FIG. 6C schematically depicts a sectional view of the catheter assembly of FIGS. 6A and 6B with the delivery catheter and the working catheter decoupled, according to one or more embodiments shown and described herein;

FIG. 7A schematically depicts a two catheter system, according to one or more embodiments shown and described herein; and

FIG. 7B schematically depicts a two catheter system, according to one or more embodiments shown and described herein.

### DETAILED DESCRIPTION

Embodiments described herein are directed to devices and methods for forming a fistula. In some embodiments, the devices and methods may be used to form a fistula between two blood vessels. More particularly, a catheter may be placed in each of two adjacent blood vessels to form a fistula therebetween with the catheters.

Fistula forming elements may be mounted to catheters which may then be used to form a fistula between vessels. However, flexibility of the catheters, spacing of vessels, thickness of the vessel walls, and/or the tortuous anatomy of the vessels, may make it difficult to provide sufficient coaptation and/or alignment between vessels for fistula formation. The embodiments described herein address the one or more aforementioned limitations. In particular, the devices and methods for forming a fistula described herein may include a catheter assembly having a delivery catheter and a working catheter that may be selectively coupled. The delivery catheter includes a delivery catheter body and an inner lumen. The working catheter includes a working catheter body and a modification device, such as an electrode. The modification device may be configured to project from a working site of the working catheter and define an active side of the working catheter. The working catheter extends through the inner lumen of the delivery catheter. When coupled, the working catheter and the delivery catheter may be simultaneously manipulated in a blood vessel. When decoupled, the working catheter and the delivery catheter may be independently manipulated in a blood vessel. At least a portion of the working catheter is more flexible than the delivery catheter. More particularly, the limited flexibility of the delivery catheter may inhibit the ability to align and coapt the working site at a site to form a fistula when the working catheter and delivery catheter are coupled. When the working catheter and the delivery catheter are decoupled, the increased flexibility of the working catheter may promote alignment and coaptation of the working site with a site to form a fistula. Various embodiments will now be described in greater detail below with reference to the figures.

As used herein, the term “proximal” means closer to or in the direction of an origin of an element, such as a catheter. The origin of a catheter may be a handle or other user-manipulated portion of the catheter. The term “distal” mean further from the origin, or handle, of the catheter. Put another way, the term “distal” mean closer to or in the direction of a tip of a catheter, which is separated from a handle of the catheter by the length of the catheter body.

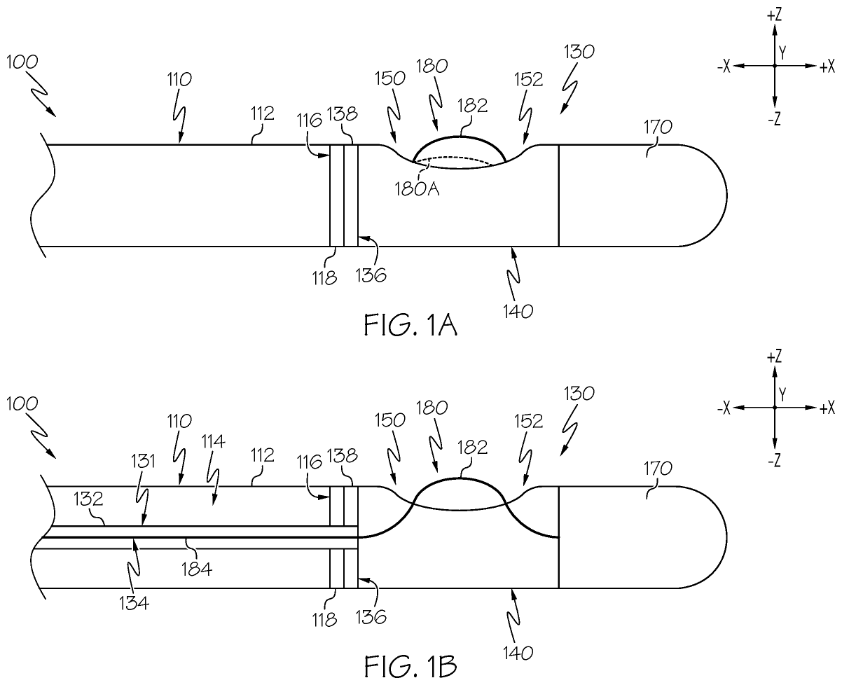

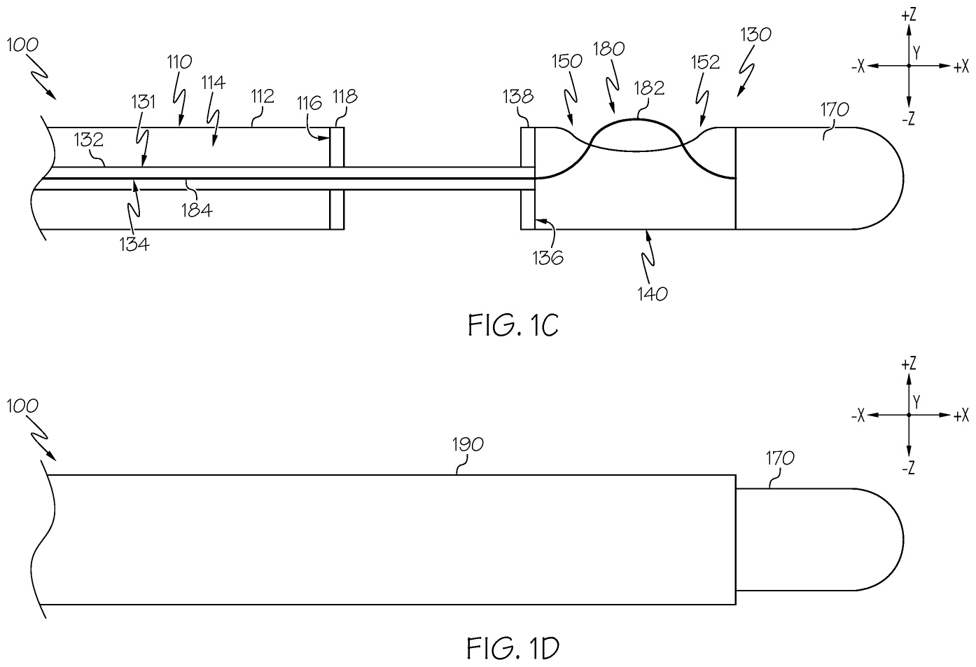

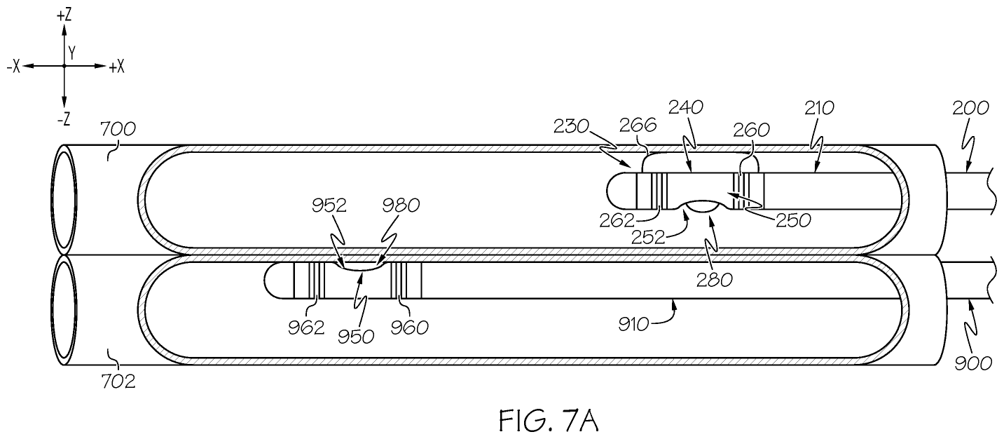

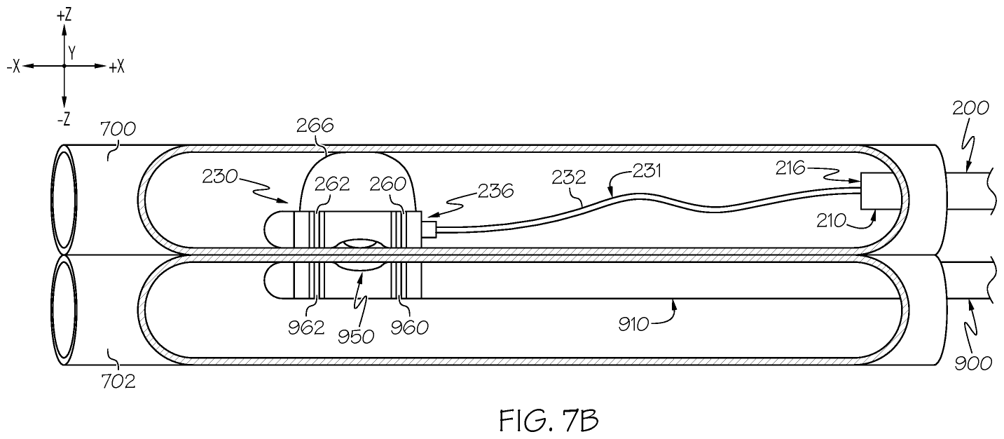

Referring now to FIGS. 1A-1C, a catheter assembly 100 (e.g., a first catheter) of a system for forming a fistula is depicted. As will be described in greater detail herein, the system may further include a second catheter 900 (FIGS. 7A and 7B). While the structure of the catheter assembly 100 will be discussed in detail, it should be appreciated that the structure of the second catheter 900 (FIGS. 7A and 7B) may mirror the catheter assembly 100 except where noted. Still referring to FIGS. 1A-1C, the catheter assembly 100 generally includes a delivery catheter 110 having a delivery catheter body 112. The delivery catheter 110 may have any desirable cross-sectional shape and any suitable diameter for intravascular use so long as the delivery catheter 110 is able to receive and house at least a portion of a working catheter 130 within, as described in further detail below. The delivery catheter 110 may further include one or more lumens 114 or other passageways extending at least partially along or through the delivery catheter body 112. For instance, the one or more lumens 114 may extend at least partially longitudinally through the delivery catheter body 112 in the direction of the x-axis of the coordinate axes of FIGS. 1A-1C.

The delivery catheter body 112 may include one or more materials to provide the delivery catheter 110 with a high degree of pushability. For instance, the delivery catheter body 112 may include one or more polymers, such as polyether ether ketone, or the like. In having a high degree of pushability, the delivery catheter 110 may be advanced axially through a blood vessel with relative ease. That is, an axial force applied at a proximal end of the delivery catheter 110 (e.g. at a handle of the delivery catheter 110) is transmitted through the delivery catheter body 112, resulting in axial advancement of the delivery catheter 110 through a blood vessel. The delivery catheter body 112 may also include a braided shaft to enhance the torsional stiffness of the delivery catheter 110. By having a high torsional stiffness, rotation applied to the proximal end of the delivery catheter 110 directly results in rotation of the distal end of the delivery catheter 110. The high degree of pushability and torsional stiffness of the delivery catheter 110 may allow a user to be able to position the delivery catheter 110 at a desirable longitudinal location within a blood vessel and orient the delivery catheter 110 such that a particular portion of the delivery catheter is facing toward a target vessel wall with some accuracy. However, the high degree of pushability and torsional stiffness of the delivery catheter 110 also reduces the flexibility of the delivery catheter 110. This reduced flexibility in turn reduces the ability of the delivery catheter 110 to coapt with a blood vessel wall and/or a second catheter in an adjacent blood vessel, as will be explained with greater detail with respect to FIGS. 7A and 7B.

The catheter assembly 100 further includes a working catheter 130. The working catheter 130 includes a working catheter body 131. The working catheter body 131 may have any desirable cross-sectional shape and any suitable diameter for intravascular. The working catheter body 131 may generally include a first portion 132 axially received within the delivery catheter 110. The working catheter body 131 may include a distal tip 170 that is particularly configured to aid in advancement of the catheter assembly 100 and/or the working catheter 130 through a blood vessel. For example, the distal tip 170 may be pointed and/or atraumatic for advancement through a blood vessel. The working catheter 130 may further include one or more lumens 134 or other passageways extending at least partially along or through the working catheter body 131. For instance, the one or more lumens 134 may extend at least partially longitudinally through the working catheter body 131 in the direction of the x-axis of the coordinate axes of FIGS. 1A-1C.

The working catheter 130 may be formed of one or more materials that insulate a lead wire 184 within the working catheter body 131 and provide the working catheter 130 with a high degree of flexibility. For instance, the working catheter body 131 may include polytetrafluoroethylene and/or polyether block amide. The high degree of flexibility of the working catheter 130 allows at least a portion of the working catheter 130 to be more easily biased against a target vessel wall as opposed to a catheter with a high degree of stiffness. In other words, the high degree of flexibility of the working catheter 130 allows the working catheter 130 to coapt with a blood vessel wall and/or a second catheter in an adjacent blood vessel. More specifically, at least a portion of the working catheter 130 is more flexible than the delivery catheter 110. That is, when the delivery catheter 110 is in a relaxed state such that the longitudinal centerline (e.g. in the direction of the x-axis of the coordinate axes of FIGS. 1A-1C) of the delivery catheter 110 is parallel with the x-axis of the coordinate axes of FIGS. 1A-1C, a force applied to the delivery catheter 110 normal to the x-axis of the coordinate axes of FIGS. 1A-1C results in a first angular deflection of the longitudinal centerline of the delivery catheter 110 from the x-axis of the coordinate axes of FIGS. 1A-1C. When the working catheter 130 is in a relaxed state such that the longitudinal centerline (e.g. in the direction of the x-axis of the coordinate axes of FIGS. 1A-1C) of the working catheter 130 is parallel with the x-axis of the coordinate axes of FIGS. 1A-1C, the same force applied to the working catheter 130 normal to the x-axis of the coordinate axes of FIGS. 1A-1C results in a second angular deflection of the longitudinal centerline of the working catheter 130 from the x-axis of the coordinate axes of FIGS. 1A-1C, where the second angular deflection is greater than the first angular deflection. Specifically, in embodiments, the first portion 132 of the working catheter body 131 is more flexible than the delivery catheter body 112.

The working catheter 130 may further include a working site 150. The working site 150 may be positioned distally (e.g. in the direction of the +x axis of the coordinate axes of FIGS. 1A-1C) of the first portion 132 of the working catheter body 131. The working site 150, as described herein, refers to a portion of the working catheter 130 positioned along the working catheter body 131 that is configured to modify a blood vessel (e.g., cut, ablate, etc.). In particular, in embodiments of the present disclosure, the working site 150 is configured to form one or more fistulas between a first blood vessel 700 (FIGS. 7A and 7B) and a second blood vessel 702 (FIGS. 7A and 7B). In embodiments, the working site 150 may be positioned along the working catheter body 131 at a position proximal to (e.g. in the −x direction of the coordinate axes of FIGS. 1A-1C) the distal tip 170. The working site 150 defines an active side 152. The working site 150 may comprise one or more openings in the active side 152 of the working site 150 that allow for passage of one or more instruments into and/or out of the working catheter body 131 for modifying the vessel. For example, the active side 152 of the working site 150 is the portion of the working site 150 that abuts or faces the area of the vessel where a modification is to be made. For instance, an electrode 180 may protrude from the active side 152 of the working site 150 and extend radially away from a longitudinal centerline of the working catheter 130 to contact a wall of the blood vessel 700 (see e.g., FIG. 7B).

Still referring to FIGS. 1A-1C, the electrode 180 may include an exposed ablation surface 182, which may be activated to ablate tissue, and a lead wire 184 or other conductor attached thereto. Particularly, when activated, current may be supplied to and/or carried from tissue and fluid via the ablation surface 182 to facilitate ablation or vaporization of tissue to form a fistula. In some embodiments, the electrode 180 may be a spring wire or leaf spring electrode, which may be movable between a retracted configuration, in which the electrode 180 is retained within the working catheter 130, and a protruding configuration, in which electrode 180 projects from a surface of the working catheter body 131, such as the working site 150. The electrode 180 may or may not be naturally biased to project from the working catheter body 131. When the electrode 180 is naturally biased to project from the working catheter body 131, a structure, such as a sleeve 190 (FIG. 1D), may be used to hold or maintain the electrode 180 in a retracted configuration, depicted by the dashed line 180A in FIG. 1A, until deployment is desired. In some embodiments, the working catheter body 131 may comprise one or more insulating materials (not shown) which may shield or otherwise protect the working catheter 130 and its components from heat generated by the electrode 180 during use. In some embodiments, a user may manipulate the electrode 180 to project the electrode 180 radially away from the working site 150 and working catheter body 131. For instance, the user may advance the lead wire 184 distally within the lumen 134 of the working catheter body 131 to cause the electrode to buckle and radially arch away from the working catheter body 131. While embodiments including the electrode 180 are discussed in detail herein, it is noted that other cutting or modification devices are contemplated and possible. For instance, ultrasonic cutting elements, laser, knives, etc. may be used in place of or in addition to the electrode 180. “Modification device,” as used herein, generally refers to any cutting or modification device, including the electrode 180, that may be used to modify (e.g. cut, ablate) a vessel wall.

The working catheter 130 further includes a non-active side 140 or region positioned opposite the active side 152 of the working site 150. For example, the non-active side 140 refers to the side of the working catheter 130 devoid of cutting and/or ablation means. The non-active side 140 of the working catheter 130 extends across the working catheter body 131 and the working site 150. In other words, the working catheter body 131 and the working site 150 may both define the non-active side 140. The non-active side 140 of the working catheter body 131 and working site 150 is diametrically opposite the active side 152 of the working site 150. Therefore, the non-active side 140 of the working catheter body 131 and working site 150 is positioned opposite the electrode 180. In other words, the non-active side 140 of the working catheter body 131 and working site 150, does not abut or face a modification being formed in the blood vessel 700 (FIG. 7B) via the active side 152 of the working site 150. Instead the non-active side 140 of the working catheter body 131 may be spaced from a modification being formed in the blood vessel 700 (FIG. 7B) by the electrode 180 by the diameter or height (e.g. in the direction of the z-axis of the coordinate axes of FIGS. 1A-1C) of at least a portion of the working catheter body 131 and/or working site 150.

Still referring to FIGS. 1A-1C, the delivery catheter 110 includes a docking site 116, and the working catheter 130 includes a nesting site 136. The docking site 116 may be positioned at a distal end of the delivery catheter 110. The nesting site 136 of the working catheter 130 may be positioned at a distal end of the first portion 132 of the working catheter body 131 and proximal the working site 150 and the electrode 180. Interaction between the docking site 116 of the delivery catheter 110 and the nesting site 136 of the working catheter 130 selectively couples the working catheter 130 with the delivery catheter 110. In the embodiment depicted in FIGS. 1A-1C, the docking site 116 of the delivery catheter 110 includes a docking magnet 118, and the nesting site 136 of the working catheter 130 includes a nesting magnet 138. The docking magnet 118 of the delivery catheter 110 may be configured to mate with the nesting magnet 138 of the working catheter, and vice versa, such that the delivery catheter 110 and the working catheter 130 may be coupled. As used herein, the term “mate” may be understood to mean a mutual attraction between a first magnet or array of magnets and a second magnet or array of magnets. The mutual attraction between the docking magnet 118 of the delivery catheter 110 and the nesting magnet 138 of the working catheter 130 may be referred to herein as a coupling force. The docking magnet 118 and the nesting magnet 138 may include any suitable combination of ferromagnetic, permanent, and/or other suitable kinds of magnets.

When the delivery catheter 110 and the working catheter 130 are coupled (as shown in FIGS. 1A and 1B) via the coupling force between docking magnet 118 of the delivery catheter 110 and the nesting magnet 138 of the working catheter 130, the delivery catheter 110 and the working catheter 130 of the catheter assembly 100 may be simultaneously manipulated. For instance, advancement of the delivery catheter 110 through a blood vessel simultaneously advances the working catheter 130 coupled to the delivery catheter 110 through the blood vessel. Similarly, when coupled, rotation of the delivery catheter 110 in a blood vessel simultaneously rotates the working catheter 130 in the blood vessel. Also, when coupled, retraction of the delivery catheter 110 through a blood vessel simultaneously retracts the working catheter 130 coupled to the delivery catheter 110 through the blood vessel.

The working catheter 130 may be decoupled from the delivery catheter 110 (as shown in FIG. 1C) to allow the working catheter 130 to be manipulated in a blood vessel independently of the delivery catheter 110. For instance, while maintaining the delivery catheter 110 in a first position, a user may apply an axial force (e.g. in the direction of the +x axis of the coordinate axes of FIGS. 1A-1C) on the working catheter 130 greater than the coupling force between the docking magnet 118 of the delivery catheter 110 and the nesting magnet 138 of the working catheter 130. In doing so, the docking site 116 of the delivery catheter 110 and the nesting site 136 of the working catheter 130, and more particularly, the docking magnet 118 of the delivery catheter 110 and the nesting magnet 138 of the working catheter 130 may be decoupled. Similarly, while maintaining the working catheter 130 in a first position, a user may apply an axial force (e.g. in the direction of the −x axis of the coordinate axes of FIGS. 1A-1C) on the delivery catheter 110 greater than the coupling force between the docking magnet 118 and the nesting magnet 138 to decouple the docking magnet 118 and the nesting magnet 138. In yet another example, a user may simultaneously apply an axial force (e.g. in the direction of the +x axis of the coordinate axes of FIGS. 1A-1C) on the working catheter 130 and an axial force (e.g. in the −x direction of the coordinate axes of the FIGS. 1A-1C) on the delivery catheter 110 to decouple the docking magnet 118 and the nesting magnet 138. The axial forces may be applied to the working catheter 130 and the delivery catheter 110 through distinct handles. That is, the working catheter 130 and the delivery catheter 110 may have separate handles that be independently manipulated by a user to apply distinct axial forces to the working catheter 130 and delivery catheter 110. In some embodiments, the catheter assembly 100 may have a single handle. For instance, the working catheter 130 and the delivery catheter 110 may extinguish at their proximal ends at a shared handle. The shared handle may include triggers, slides, and the like, that upon actuation, may apply axial force independently to the working catheter 130 and/or the delivery catheter 110.

Once decoupled, an axial force (e.g. in the direction of the +x axis of the coordinate axes of FIGS. 1A-1C) applied to the working catheter 130 may advance the working catheter 130 in a blood vessel while the delivery catheter 110 remains at its longitudinal positon in the blood vessel when decoupling of the working catheter 130 and the delivery catheter 110 occurred. It should be appreciated that the delivery catheter 110 may also be manipulated independently of the working catheter 130 when the delivery catheter 110 and the working catheter 130 are decoupled. For instance, once decoupled, a user may apply an axial force (e.g. in the direction of the −x axis of the coordinate axes of FIGS. 1A-1C) to the delivery catheter 110 to withdraw the delivery catheter 110 in a blood vessel while the working catheter 130 remains at its longitudinal position in the blood vessel when decoupling of the working catheter 130 and the delivery catheter 110 occurred. Moreover, it should be appreciated that once decoupled, a user may manipulate the working catheter 130 and/or the delivery catheter 110 to bring the nesting site 136 and docking site 116 into contact and re-couple the working catheter 130 and delivery catheter 110.

As noted above, the delivery catheter 110 is sized and shaped to be able to receive at least a portion of the working catheter 130 therein. In other words, the lumen 114 of the delivery catheter 110 has a diameter that is greater than the outer diameter of at least a portion of the working catheter 130. Particularly, in the embodiment depicted in FIGS. 1A-1C, the diameter of the lumen 114 of the delivery catheter body 112 is greater than the diameter of the first portion 132 of the working catheter body 131. In some embodiments, the diameter of the lumen 114 of the delivery catheter 110 may be defined by the outer surfaces of the delivery catheter body 112. In other embodiments, the diameter of the lumen 114 may be less than a diameter defined by the outer surfaces of the delivery catheter body 112. While diameters of the delivery catheter 110 and the working catheter 130 are particularly discussed in the foregoing and below description, it should be appreciated that the term may be interchangeable with height and width depending on the cross-sectional shape of the component being described. That is, in embodiments a height (e.g. in the direction of the z-axis of the coordinate axes of FIGS. 1A-1C) and a width (e.g. in the direction of the y-axis of the coordinate axes of FIGS. 1A-1C) of the inner lumen 114 of the delivery catheter body 112 may be greater than the height and width of the first portion 132 of the working catheter body 131 such that the first portion 132 of the working catheter body may be received within the inner lumen 114 of the delivery catheter body 112.

The docking magnet 118 of the delivery catheter 110 may have any desirable cross-sectional shape and any suitable diameter. In embodiments, the docking magnet 118 may have the same cross-sectional shape and diameter as the delivery catheter body 112 such that the outer surface of the delivery catheter body 112 is predominantly flush with the outer surface of the docking magnet 118. In embodiments, the docking magnet 118 may be within the delivery catheter body 112 such that the distal edge of the docking magnet 118 is housed within the distal edge of the delivery catheter body 112. The docking magnet 118 may be annular and include an internal aperture that allows passage of the first portion 132 of the working catheter body 131 therethrough.

The nesting magnet 138 of the working catheter 130 may have any desirable cross-sectional shape and any suitable diameter. In embodiments, the nesting magnet 138 may have the same cross-sectional shape and diameter as the docking magnet 118 such that the outer surface of the nesting magnet 138 is predominantly flush with the outer surface of the docking magnet 118 when the working catheter 130 is coupled with the delivery catheter 110. The nesting magnet 138 may have the same cross-sectional shape and diameter as the working site 150 of the working catheter 130 such that the outer surface of the nesting magnet 138 is predominantly flush with the outer surface of the working site 150. The nesting magnet 138 may have a smaller diameter than the working site 150. The nesting magnet 138 may have a smaller diameter than the docking magnet 118. The nesting magnet 138 may be annular and include an internal aperture that allows passage of at least the lead wire 184 therethrough. The internal aperture of the nesting magnet 138 may also allow passage of the first portion 132 of the working catheter body 131 therethrough.

The working site 150 of the working catheter 130 may have any desirable cross-sectional shape and any suitable diameter. In embodiments, the working site 150 may have the same cross-sectional shape and diameter as the nesting magnet 138, the docking magnet 118, and/or the delivery catheter body 112 such that the working site 150 is predominantly flush with the outer surface of the delivery catheter body 112 when the working catheter 130 is coupled with the delivery catheter 110. The working site 150 may have the same cross-sectional shape and/or diameter as the docking magnet 118 such that the working site 150 is predominantly flush with the outer surface of the docking magnet 118 when the working catheter 130 is coupled with the delivery catheter 110. The working site 150 may have a larger diameter than the delivery catheter body 112, the docking magnet 118, and/or the nesting magnet 138. The working site 150 of the working catheter 130 may have a larger diameter than the first portion 132 of the working catheter body 131. In embodiments, the working site 150 is sized relative to the delivery catheter body 112 and lumen 114, and positioned relative to the nesting site 136 such that the working site 150 remains exposed from the delivery catheter 110 when the delivery catheter 110 and working catheter 130 are coupled.

In the embodiment depicted in FIGS. 1A-1C, the working catheter 130 and the delivery catheter 110 selectively couple at axial facing surfaces of the docking magnet 118 and the nesting magnet 138. This is a non-limiting example, however. For instance, in some embodiments, the docking magnet 118 may be annular, and an outer diameter of the nesting magnet 138 may be less than an inner diameter of the docking magnet 118. In such embodiments, the nesting magnet 138 may be received within the annular recess of the docking magnet 118 and the coupling force may be generated between the outer diameter of the nesting magnet 138 and the inner diameter of the docking magnet 118. In such embodiments, the docking magnet 118 may be positioned within the delivery catheter body 112, such that when the working catheter 130 and delivery catheter 110 are coupled, the nesting magnet 138 is also positioned within the delivery catheter body 112. In embodiments, the diameter of the working site 150 may be greater than the diameter of the nesting magnet 138. Therefore, when the working catheter 130 and the delivery catheter 110 are coupled such that the nesting magnet 138 is within the annular recess of the docking magnet 118, the outer surface of the working site 150 may be predominantly flush with the outer surface of the docking magnet 118 and/or the delivery catheter body 112.

In embodiments, the nesting magnet 138 may have dual functionality. That is, as explained above, in conjunction with the docking magnet 118, the nesting magnet may selectively couple the working catheter 130 and the delivery catheter 110. When the working catheter 130 and the delivery catheter 110 are decoupled, however, the nesting magnet 138 may further function to mate with one or more magnets or magnet arrays of a second catheter 900 (FIGS. 7A and 7B) in an adjacent blood vessel, thereby increasing coaptation between the working catheter 130 and the second catheter 900 (FIGS. 7A and 7B), as will be discussed in further detail with respect to FIGS. 7A and 7B.

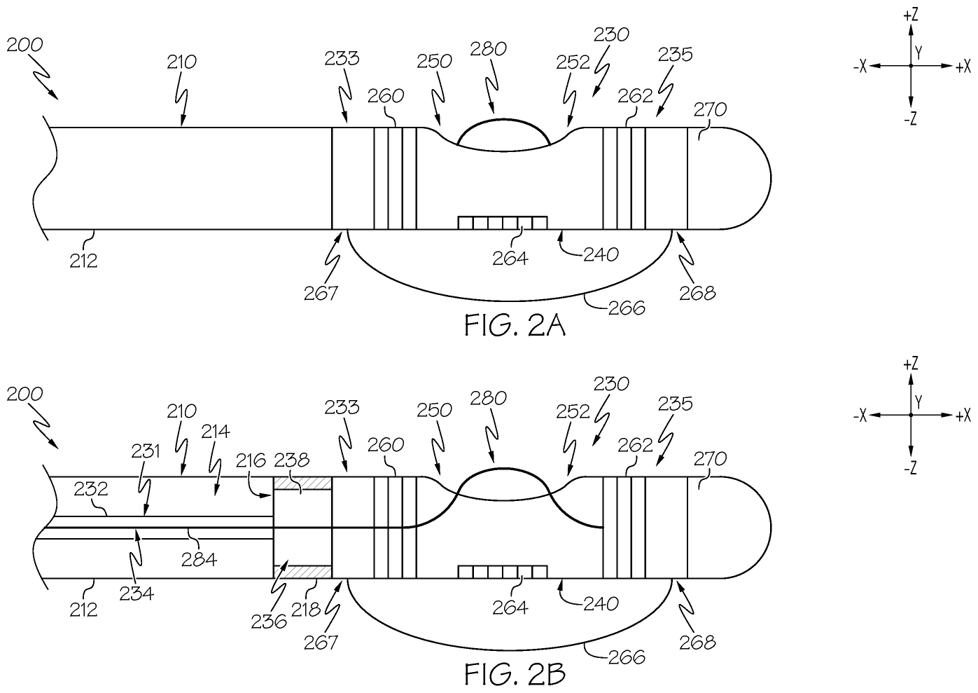

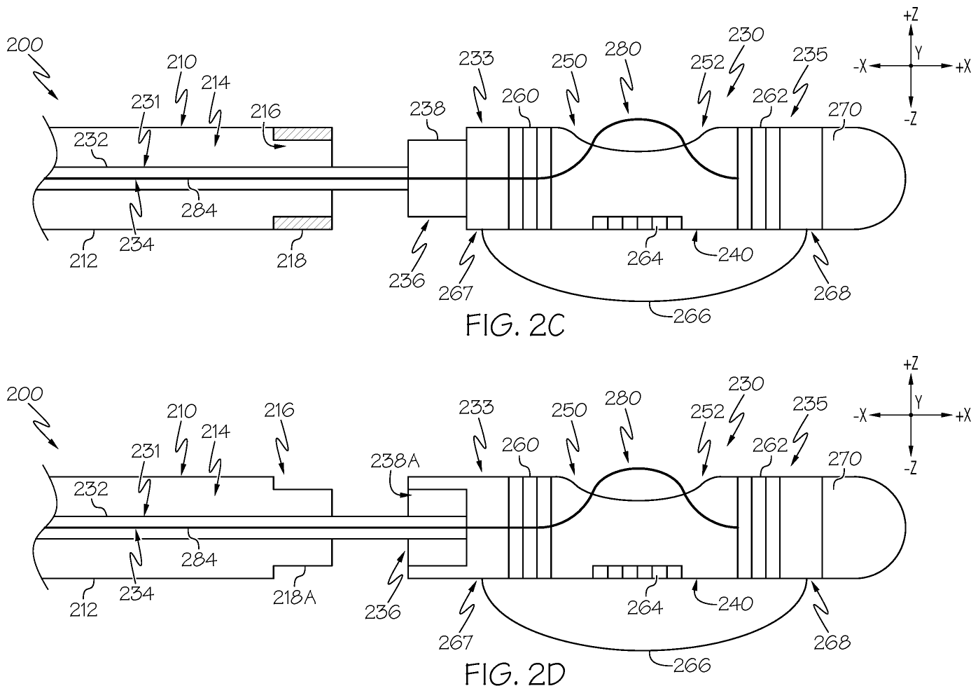

Referring now to FIGS. 2A-2C, a catheter assembly 200 is depicted. The catheter assembly 200 may resemble the catheter assembly 100 discussed in FIGS. 1A-1C in all aspects except as discussed herein. For instance, similar to the catheter assembly 100, the catheter assembly 200 generally includes a delivery catheter 210 having a delivery catheter body 212 and one or more lumens 214 or other passageways extending at least partially along or through the delivery catheter body 212. The catheter assembly 200 further includes a working catheter 230 having a working catheter body 231. A first portion 232 of the working catheter body 231 is able to be received within the delivery catheter 210. The working catheter 230 may further include one or more lumens 234 or other passageways extending at least partially along or through the working catheter body 231. At least a portion of the working catheter 230 is more flexible than the delivery catheter 210. Particularly, the first portion 232 of the working catheter body 231 may be more flexible than the delivery catheter 210. The working catheter 230 further includes a working site 250 that allows for passage of one or more instruments, such as the electrode 280, into and/or out of the working catheter body 231. The working catheter 230 of the catheter assembly 200 further includes a nesting site 236, and the delivery catheter 210 further includes a docking site 216, which allow for selectively coupling the working catheter 230 with the delivery catheter 210.

Unlike the working catheter 130 of the catheter assembly 100, the working catheter body 231 of the working catheter 230 includes a second portion 233 and a third portion 235. The second portion 233 is positioned distal (e.g. in the direction of the +x axis of the coordinate axes of FIGS. 2A-2C) the first portion 232 and proximal the working site 250. In embodiments, the second portion 233 is positioned distal the nesting site 236 and proximal (e.g. in the direction of the −x axis of the coordinate axes of FIGS. 2A-2C) the working site 250. The third portion 235 is positioned distal the working site 250 and proximal a tip 270 of the working catheter 230. The second portion 233 and the third portion 235 may each have a greater diameter than the first portion 232 of the working catheter 230. In embodiments, the second portion 233 and/or the third portion 235 of the working catheter body 231 may have roughly the same diameter as the working site 250, such that the outer surfaces of the second portion 233 and/or the third portion 235 are predominantly flush with the outer surface of the working site 250. In embodiments, the second portion 233 and/or the third portion 235 of the working catheter body 231 may have roughly the same diameter as the delivery catheter body 212. In such embodiments, the outer surface of the second portion 233 may be predominantly flush with the delivery catheter body 212 when the working catheter 230 is coupled with the delivery catheter 210. In embodiments, the second portion 233, working site 250, and the third portion 235 of the working catheter 230 are sized relative to the delivery catheter body 212 and lumen 214, and positioned relative to the nesting site 236 such that the second portion 233, working site 250, and third portion 235 of the working catheter 230 remain exposed from the delivery catheter 210 when the delivery catheter 210 and working catheter 230 are coupled. It should be appreciated that in embodiments, the working catheter 230 may include only one of the second portion 233 or the third portion 235. For instance, in embodiments, the second portion 233 may be positioned distal the first portion 232 and proximal the working site 250 while the tip 270 of the working catheter 230 may be positioned distally adjacent the working site 250 without the presence of third portion 235.

Still referring to FIGS. 2A-2C, the working catheter 230 may include one or more arrays of magnets arranged longitudinally along the working catheter body 231. For instance, the working catheter 230 may include a first array of magnets 260 that extends longitudinally along at least a portion of the second portion 233 of the working catheter body 231. The working catheter 230 may include a second array of magnets 262 that extends longitudinally along at least a portion of the third portion 235 of the working catheter body 231. In embodiments, the working catheter 230 may include a third array of magnets 264 positioned in the working site 250 behind an active side 252 of the working site 250. More particularly, the third array of magnets 264 may be positioned along and/or within a non-active side 240 of the working site 250. In embodiments, the working catheter 230 may include the first array of magnets 260, the second array of magnets 262, and the third array of magnets 264 individually or in any combination. It should be appreciated that while the phrase “array of magnets” is used herein, that each of the arrays of magnets 260, 262, and 264 may be configured as a single magnet along the working catheter body 231 and/or working site 250.

The arrays of magnets 260, 262, and 264 described herein may be permanent magnets comprising one or more hard magnetic materials, such as but not limited to alloys of rare earth elements (e.g., samarium-cobalt magnets or neodymium magnets, such as N52 magnets) or alnico. In some variations, the arrays of magnets 260, 262, and 264 may comprise anisotropic magnets; in other variations, the arrays of magnets 260, 262, and 264 may comprise isotropic magnetics. In some variations, the arrays of magnets 260, 262, and 262 may be formed from compressed powder. In some variations, a portion of the arrays of magnets 260, 262, and 264 (e.g., a permeable backing) may comprise one or more soft magnetic materials, such as but not limited to iron, cobalt, nickel, or ferrite. It should be appreciated that in systems comprising two catheters, either the working catheter 230 or the second catheter 900 (FIGS. 7A and 7B) may comprise ferromagnetic elements (i.e., elements attracted to but not generating a permanent magnetic field). For example, in some variations, the working catheter 230 may include only one or more ferromagnetic elements while the second catheter 900 (FIGS. 7A and 7B) may comprise one or more permanent magnets. In other variations, the second catheter 900 (FIGS. 7A and 7B) may include only one or more ferromagnetic elements while the working catheter 230 may comprise one or more permanent magnets. However, in other variations, one or both of the working catheter 230 and the second catheter 900 (FIGS. 7A and 7B) may include any suitable combination of ferromagnetic, permanent, and/or other suitable kinds of magnets.

Generally, the dimensions of the arrays of magnets 260, 262, and 264 described herein may be selected based upon the size of the working catheter 230 carrying the arrays of magnets 260, 262, and 264, which in turn may be selected based upon the anatomical dimensions of the selected blood vessels through which the working catheter 230 may be advanced. For example, if the working catheter 230 is to be advanced through a blood vessel 700 (FIGS. 7A and 7B) having an internal diameter of about 3 mm, it may be desirable to configure any array of magnets 260, 262, and 264 to be less than about 3 mm at the widest part of their cross-sections, to reduce the risk of injury to vessel walls during advancement and manipulation of the working catheter 230. Each array of magnets 260, 262, and 264 may have any suitable length. In some variations, the arrays of magnets 260, 262, and 264 may include a plurality of square magnets having square cross sections. In other embodiments, each magnet of the arrays of magnets 260, 262, and 264 may have any suitable shape for placement inside or outside of the catheter. The magnets may be cylindrical, semi-cylindrical, tube-shaped, box-shaped, or the like.

In embodiments, the outer surfaces of the arrays of magnets 260, 262, and 264 may be flush or in line with the outer surface of the second portion 233, third portion 235, and working site 250, respectively. In other embodiments, the magnets 260, 262, and 264 may be positioned radially within the working catheter body 231 away from the outer surface of the working catheter body 231. In other embodiments the outer surfaces of the arrays of magnets 260, 262, and 264 may extend a distance radially beyond the outer surface of the second portion 233, third portion 235, and working site 250, respectively. Each array of magnets 260, 262, 264 may be fixed in or on the working catheter 230 by any suitable method. For example, in some variations the one or more arrays of magnets 260, 262, and 264 may be embedded in, adhered to, or friction-fit within the working catheter 230.

In some embodiments, the working catheter 230 may include one or more biasing rails. While embodiments including a single biasing rail are discussed in detail herein, it should be readily appreciated that the working catheter 230 may include two, three, or more biasing rails. In embodiments the working catheter 230 may include a biasing rail 266. The biasing rails 266 may extend longitudinally (e.g. in the direction of the x-axis of the coordinate axes of FIGS. 2A-2C) along a length of the working catheter body 231. The biasing rail 266 may arch radially away from the working catheter body 231 between a proximal point 267 and a distal point 268 of the working catheter body 231. The proximal point 267 may be positioned proximal a first end of at least one array of the arrays of magnets 260, 262, 264, and the distal point 268 may be positioned distal a second end of the at least one array of the arrays of magnets 260, 262, 264. Accordingly, the biasing rail 266 may be configured to longitudinally span the at least one array of the arrays of magnets 260, 262, 264. In other words, at least one of the first array of magnets 260, the second array of magnets 262, and the third array of magnets 264 may be positioned longitudinally between the proximal point 267 and the distal point 268. In embodiments, the proximal point 267 is positioned along the second portion 233 of the working catheter body 231, and the distal point 268 is positioned along the third portion 235 of the working catheter body 231. In embodiments the biasing rail 266 may be arranged to bias the one or more arrays of magnets 260, 262, 264 against a blood vessel wall, as will be discussed in further detail with respect to FIGS. 7A and 7B. It should be appreciated, however, that the biasing rail 266 may be included on the working catheter 230 without the one or more arrays of magnets 260, 262, 264.

In embodiments, the biasing rail 266 may extend radially from the non-active side 240 of the working catheter 230. In other words, the proximal point 267 and distal point 268 may be positioned along the non-active side 240 of the working catheter body 231. In embodiments, the working site 250 may be positioned longitudinally between the proximal point 267 and the distal point 268. In embodiments, the biasing rail 266 may be configured to bias the working site 250, and more specifically the active side 252 of the working site 250, against a blood vessel wall, as will be discussed in further detail with respect to FIGS. 7A and 7B.

In embodiments the biasing rail 266 may be fixedly secured to the working catheter body 231 at the proximal point 267 and the distal point 268. In such embodiments, the biasing rail 266 may be fixed to the working catheter body 231 at the proximal point 267 and the distal point 268 with a suitable polymer or adhesive, such as glue, laser welding, heat shrunk plastic wrap, and the like.

In embodiments, the biasing rail 266 may have a circular cross section. In embodiments, the biasing rail 266 may be a flat ribbon having a substantially rectangular cross section. In some embodiments, the biasing rail 266 may transition between a first section of the biasing rail 266 having a circular cross section and a second section of the biasing rail 266 having a substantially rectangular cross section. For instance, a proximal end of the biasing rail 266 adjacent to the proximal point 267 may have a circular cross section, a distal end of the biasing rails 266 adjacent the distal point 268 may have a circular cross section, and a section of the biasing rail 266 between the proximal and distal ends may have a rectangular cross section. In embodiments, the biasing rail 266 may be made of metal, plastic, polymer, metal coated in plastic, a composite of any of said materials, and the like. For instance, the biasing rail 266 may be nitinol, stainless steel, polyethylene terephthalate, polyether ether ketone, polytetrafluoroethylene, polyimide, and the like. The biasing rail 266 may be a material that exhibits shape memory and returns to a set or desired shape.

In some embodiments, the biasing rail 266 may expand from a low-profile configuration to an extended configuration. In the low-profile configuration, the biasing rail 266 may be positioned in a non-contacting state, in which the biasing rail 266 does not apply a biasing force to the wall of the blood vessel 700 (FIGS. 7A and 7B) to bias the working catheter 230 laterally within the blood vessel 700 (FIGS. 7A and 7B). In the low-profile configuration, the biasing rail 266 may be maintained substantially flush with or within the working catheter body 231. As such, the working catheter 230 may be advanced to a desired location within the blood vessel 700 (FIGS. 7A and 7B) without the biasing rail 266 extending radially from the working catheter body 231 and applying a biasing force to the wall of the blood vessel 700 (FIGS. 7A and 7B) to bias the working catheter 230 laterally within the blood vessel 700 (FIGS. 7A and 7B). In the extended configuration, at least a portion of the biasing rail 266 may radially extend outward from the outer surface of the working catheter body 231 to be in a contacting state, in which at least a portion of the biasing rail 266 applies a biasing force to the wall of the blood vessel 700 (FIGS. 7A and 7B). Accordingly, a maximum distance of radial deflection of the biasing rail 266 from the outer surface of the working catheter body 231, when in the extended configuration, may be greater than the maximum distance of radial deflection of the biasing rail 266 from the outer surface of the working catheter body 231, when in the low-profile configuration.

In embodiments, the biasing rail 266 described herein may be biased toward the extended configuration. That is, the biasing rail 266 may be configured to self-expand from the low-profile configuration to the extended configuration. Put yet another way, the biasing rail 266 may be in its natural resting state in the extended configuration, with the biasing rail 266 radially extending a predetermined distance away from the outer surface of the working catheter body 231. In such embodiments, a force may be required to hold the biasing rail 266 in the low-profile configuration. For instance, a sleeve 190 (such as illustrated in FIG. 1D) may be advanced distally to maintain the biasing rail 266 in the low-profile configuration. With the sleeve 190 (such as illustrated in FIG. 1D) advanced distally over the working catheter body 231, the biasing rail 266 may be compressed by the sleeve 190 (such as illustrated in FIG. 1D) against the working catheter body 231. In other words, the biasing rail 266 may be compressed and maintained in the low-profile configuration within the space between the working catheter body 231 and the sleeve 190 (such as illustrated in FIG. 1D). The sleeve 190 (such as illustrated in FIG. 1D) may be retracted in the proximal direction, thereby exposing the biasing rail 266. With the sleeve 190 (such as illustrated in FIG. 1D) no longer applying a force to the biasing rail 266 to maintain the biasing rail 266 in the low-profile configuration, and due to the natural bias of the biasing rail 266, the biasing rail may naturally expand into the extended configuration, as shown in FIGS. 2A-2C.

In some embodiments, the biasing rail 266 may be made of a shape-memory alloy, such as copper-aluminum-nickel and nickel-titanium, that changes shape due to environmental cues, such as temperature. For instance, the active shape of the biasing rail 266 may be the extended configuration depicted in FIGS. 2A-2C. The transition temperature of the shape-memory alloy may be above standard room temperatures. In some embodiments, the transition temperature of the shape-memory alloy may be roughly at internal body temperature. Therefore, outside of a patient, at standard room temperature, the biasing rail 266 may deform into the low-profile configuration, in which the biasing rail 266 is in a non-contacting state and does not extend radially away from the outer surface of the working catheter body 231 to apply a biasing force to a wall of the blood vessel 700 (FIGS. 7A and 7B). As the temperature of the biasing rail 266 increases, the biasing rail 266 may naturally transition from the low-profile configuration to the extended configuration, in which the biasing rail 266 is in a contacting state and extend radially away from the outer surface of the working catheter body 231 to apply a biasing force to the wall of the blood vessel 700 (FIGS. 7A and 7B) to bias the working catheter 230 laterally within the blood vessel 700 (FIGS. 7A and 7B). In embodiments, the biasing rail 266 may transition from the low-profile configuration to the extended configuration at temperatures above 30° C., temperatures above 32° C., temperatures above 35° C., and like temperatures between standard room temperature of 20° C. and internal body temperature at 37° C.

Still referring to FIGS. 2A-2C, as noted above, the working catheter 230 of the catheter assembly 200 includes the nesting site 236, and the delivery catheter 210 includes the docking site 216, which allow for selective coupling of the working catheter 230 with the delivery catheter 210. The nesting site 236 and the docking site 216 are sized and shaped such that the nesting site 236 may be selectively friction fit with the docking site 216. That is, when joined together, the friction forces between the nesting site 236 and the docking site 216 are large enough to couple the working catheter 230 and the delivery catheter 210. When coupled (as shown in FIGS. 2A and 2B), the working catheter 230 and the delivery catheter 210 may be advanced or otherwise manipulated simultaneously in a blood vessel. The friction force between the docking site 216 and the nesting site 236 may be referred to herein as a coupling force.

As depicted, the docking site 216 may include a flange 218. The flange 218 forms a narrowed region of the lumen 214 of the delivery catheter body 212. The nesting site 236 may include a protrusion 238. The protrusion 238 may have a smaller diameter than the second portion 233, working site 250, and/or third portion 235 of the working catheter 230. The protrusion 238 has a diameter sized to fit within and form a friction fit with the narrowed region of the lumen 214 formed by the flange 218. Therefore, the protrusion 238 may be received within the narrowed region of the lumen 214. The friction between the protrusion 238 and the flange 218 couples the working catheter 230 and the delivery catheter 210. In embodiments, the delivery catheter 210 may not include the flange 218 or a narrowed region of the lumen 214. In such embodiments, the protrusion 238 may interact with the inner wall of the delivery catheter body 212 such that the friction between the protrusion 238 and the inner wall of the delivery catheter body 212 couples the working catheter 230 and the delivery catheter 210.

While the nesting site 236 of the working catheter 230 has been described as the male component and the docking site 216 of the delivery catheter 210 has been described as the female component of the friction fit interaction between the working catheter 230 and the delivery catheter 210, it should be appreciated that this is a non-limiting example. For instance, and with reference to FIG. 2D, in some embodiments, the docking site 216 of the delivery catheter 210 may include a protrusion 218A extending from the distal end of the delivery catheter body 212 and having a first diameter, and the nesting site 236 of the working catheter 230 may include an aperture 238A having a second diameter sized to receive and form a friction with the protrusion 218A of the docking site 216. Specifically, the aperture 238A of the nesting site 236 may extend at least partially longitudinally through the second portion 233 of the working catheter body 231. In yet other embodiments, the delivery catheter 210 need not include a protrusion. For instance, the aperture 238A of the nesting site 236 of the working catheter 230 may be sized to receive the delivery catheter body 212. In such embodiments, a distal end of the delivery catheter body 212 may be received within the aperture 238A of the working catheter 230. It should be appreciated that in the discussed embodiments, the protrusion of the friction fit interaction, whether a portion of the docking site 216 or the nesting site 236, includes an internal aperture that allows for passage of, at least, a lead wire 284 therethrough, and in some embodiments both the lead wire 284 and the first portion 232 of the working catheter body 231.

As in the embodiment discussed with reference to FIGS. 1A-1C, by applying an axial force to at least one of the working catheter 230 or the delivery catheter 210, a force may be imparted at the coupled docking site 216 and nesting site 236 that is greater than the coupling friction force between the docking site 216 and the nesting site 236. Accordingly, the working catheter 230 and the delivery catheter 210 may be decoupled (as shown in FIGS. 2C and 2D) and independently manipulated in a blood vessel. Moreover, by further manipulating the working catheter 230 and/or the delivery catheter 210, the working catheter 230 and delivery catheter 210 may be re-coupled by interacting the docking site 216 and the nesting site 236.

It should further be appreciated that the docking site 216 and nesting site 236 of the catheter assembly 200 may be substituted with the docking site 116 (FIGS. 1A-1C) and nesting site 136 (FIGS. 1A-1C) of the catheter assembly 100 (FIGS. 1A-1C), and vice versa. That is, the working catheter 230 and the delivery catheter 210, in embodiments, could selectively mate by means of the docking magnet 118 (FIGS. 1A-1C) and the nesting magnet 138 (FIGS. 1A-1C) instead of the friction fit assembly discussed, and vice versa. Additionally, it should be appreciated that the working catheter 130 (FIGS. 1A-1C), in embodiments, could readily include any or all of the second portion 233, third portion 235, first array of magnets 260, second array of magnets 262, third array of magnets 264, and biasing rail 266 discussed with reference to FIGS. 2A-2C.

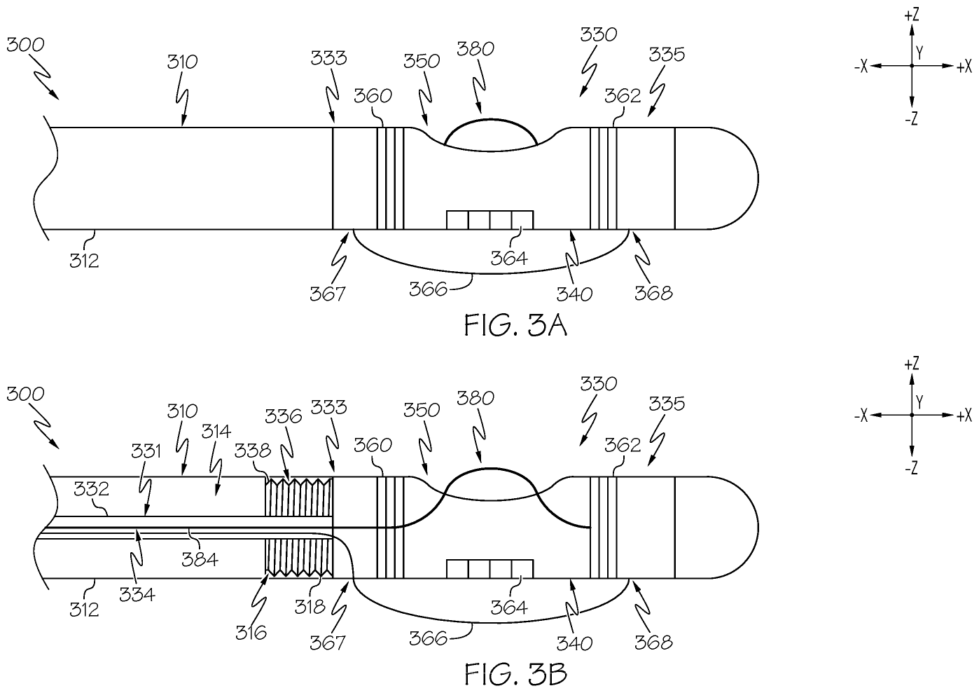

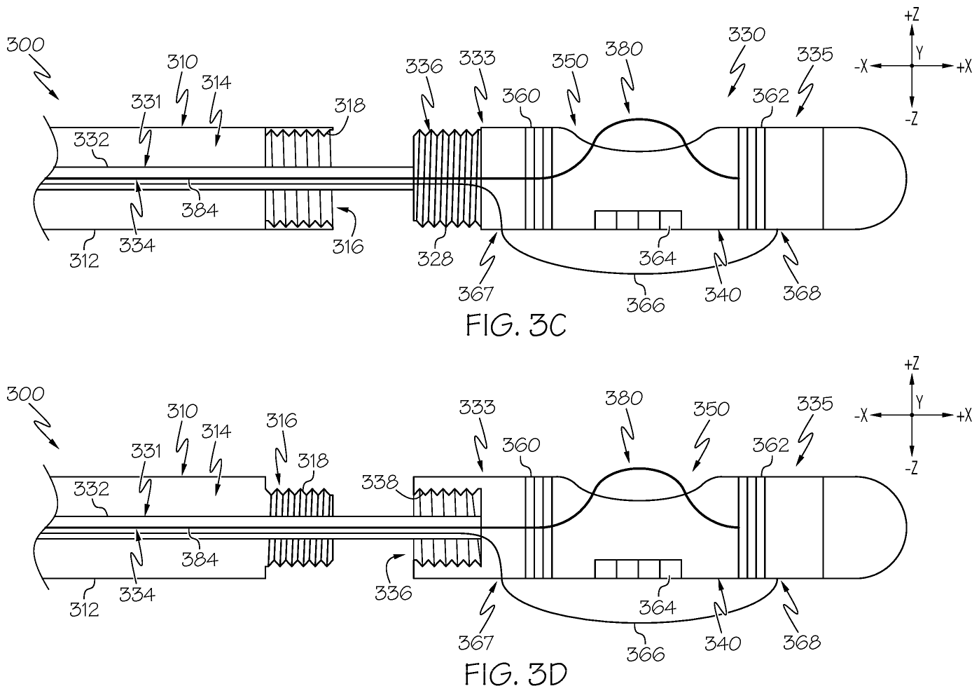

Referring now to FIGS. 3A-3D, a catheter assembly 300 is depicted. The catheter assembly 300 may resemble the catheter assembly 100 discussed in FIGS. 1A-1D and/or the catheter assembly 200 discussed in FIGS. 2A-2D in all aspects except as discussed herein. For instance, the catheter assembly 300 generally includes a delivery catheter 310 having a delivery catheter body 312 and one or more lumens 314 or other passageways extending at least partially along or through the delivery catheter body 312. The catheter assembly 300 further includes a working catheter 330 having a working catheter body 331. A first portion 332 of the working catheter body 331 is able to be received within the delivery catheter 310. The working catheter 330 may further include one or more lumens 334 or other passageways extending at least partially along or through the working catheter body 331. At least a portion of the working catheter 330 is more flexible than the delivery catheter 310. Particularly, the first portion 332 of the working catheter body 331 may be more flexible than the delivery catheter 310. The working catheter 330 further includes a working site 350 that allows for passage of one or more instruments, such as the electrode 380, into and/or out of the working catheter body 331. The working catheter 330 of the catheter assembly 300 further includes a nesting site 336, and the delivery catheter 310 further includes a docking site 316, which allow for selectively coupling the working catheter 330 with the delivery catheter 310. The working catheter 330 may include a second portion 333 and/or a third portion 335. A first array of magnets 360 may extend longitudinally along at least a portion of the second portion 333, and a second array of magnets 362 may extend longitudinally along at least a portion of the third portion 335. A third array of magnets 364 may be positioned in the working site 350. The working catheter 330 may include a biasing rail 366. The biasing rail 366 may arch radially away from the working catheter body 331 between a proximal point 367 and a distal point 368 along a non-active side 340 of the working catheter body 331.

Unlike previous embodiments discussed, the nesting site 336 and the docking site 316 are sized and shaped to allow for selective threaded coupling of the working catheter 330 and the delivery catheter 310. When coupled (as shown in FIGS. 3A and 3B), the working catheter 330 and the delivery catheter 310 may be advanced or otherwise manipulated simultaneously in a blood vessel. Referring to FIGS. 3A-3C more particularly, the docking site 316 of the delivery catheter 310 includes docking threads 318 extending from a distal end (e.g. in the direction of the +x axis of the coordinate axes of FIGS. 3A-3C) of the delivery catheter body 312 at least partially longitudinally through the delivery catheter body 312. The docking threads 318 may be female threads. The docking threads 318 may be formed along an interior surface of the delivery catheter body 312. The nesting site 336 may include nesting threads 338 that threadedly couple with the docking threads 318 of the docking site 316. The nesting threads 338 may be male threads. Accordingly, the nesting site 336 may be selectively received within the docking site 316. To selectively decouple the working catheter 330 and the delivery catheter 310, a user may hold the delivery catheter 310 stationary while rotating the working catheter 330 to disengage the nesting threads 338 from the docking threads 318. Similarly, the user may hold the working catheter 330 stationary while rotating the delivery catheter 310 to disengage the nesting threads 338 and the docking threads 318. After the nesting site 336 is passed out of the docking site 316, the working catheter 330 and the delivery catheter 310 may be independently manipulated. Moreover, once decoupled (as shown in FIGS. 3C and 3D), the working catheter 330 and/or delivery catheter may be manipulated to re-couple the working catheter 330 and delivery catheter 310 via interaction between the nesting site 336 and docking site 316. The nesting site 336 includes an internal aperture that allows for passage of, at least, a lead wire 384 of the electrode 380 therethrough, and in some embodiments both the lead wire 384 and the first portion 332 of the working catheter body 331.

While the nesting site 336 of the working catheter 330 has been described as the male component and the docking site 316 of the delivery catheter 310 has been described as the female component of the threaded interaction between the working catheter 330 and the delivery catheter 310, it should be appreciated that this is a non-limiting example. For instance, and with reference to FIG. 3D, in some embodiments, the docking site 316 of the delivery catheter 310 may extend from the distal end of the delivery catheter body 312 and have a first diameter, and the nesting site 336 of the working catheter 330 may be a threaded aperture having a second diameter greater than the first diameter to allow the docking site 316 to be received within the nesting site 336. Specifically, the nesting site 336 may extend at least partially longitudinally through the second portion 333 of the working catheter body 331. The docking site 316 includes docking threads 318, which may be male threads, and the nesting site 336 includes nesting threads 338, which may be female threads. In such embodiments, the docking site 316 includes an internal aperture that allows for passage of, at least, the lead wire 384 of the electrode 380 therethrough, and in some embodiments both the lead wire 384 and the first portion 332 of the working catheter body 331. In yet other embodiments, the docking site 316 need not extend from the distal end of the delivery catheter 310. For instance, the docking site 316 may extend longitudinally along a length of the delivery catheter body 312. In such embodiments, the docking threads 318 may be formed in the outer surface of the delivery catheter body 312, and the nesting site 336 may have a diameter greater than the diameter of the delivery catheter body 312, such that the docking site 316 may be received within the nesting site 336.

Referring to FIGS. 3A-3D, the working catheter 330 may include the biasing rail 366, which may resemble the biasing rail 266 discussed with respect to FIGS. 2A-2D, except as noted herein. Particularly, the biasing rail 366 may be configured such that the biasing rail 366 is moveable within the working catheter body 331 of the working catheter 330. For instance, the biasing rail 366 may extend through the lumen 334 of the working catheter body 331. While the biasing rail 366 is depicted as extending through the lumen 334 along with the lead wire 384, it should be appreciated that in some embodiments, the biasing rail 366 may extend through a separate lumen of the working catheter body 331 than the lead wire 384. In embodiments, the biasing rail 366 may extend through an opening of the working catheter body 331 at the proximal point 367. That is, the proximal point 367 may define an opening into the second portion 333 of the working catheter body 331. The biasing rail 366 may be coupled to the working catheter body 331 at the distal point 368. The biasing rail 366 may be fixed to the working catheter body 331 at the distal point 368 with a suitable polymer or adhesive, such as glue, laser welding, heat shrunk plastic wrap, and the like. The biasing rail 366 may extend proximally (e.g. in the −x direction of the coordinate axes of FIGS. 3A-3D) through the second portion 333 of the working catheter body 331, the nesting site 336, and the first portion 332 of the working catheter body 331. Accordingly, the biasing rail 366 may be movable within the working catheter body 331. The biasing rail 366 may axially move within the lumen 334 such that the biasing rail 366 expands outward from the opening at the proximal point 367. As the biasing rail 366 expands outward from the opening at the proximal point 367, the biasing rail 366 may radially extend away from the working catheter body 331 between the proximal point 367 and the distal point 368.

More particularly, the biasing rail 366 may be user-manipulated from a low-profile configuration to an extended configuration. For example, the biasing rail 366 may extend to a hand control, switch, actuator, or other user-manipulated device coupled to the proximal end of the biasing rail 366. Through actuation of the user-manipulated device, a user may advance the biasing rail 366 distally through the lumen 334 and/or retract the biasing rail 366 proximally through the lumen 334. As a user advances the biasing rail 366 distally, the biasing rail 366 expands outwardly from the opening at the proximal point 367.

It should further be appreciated that the docking site 316 and nesting site 336 of the catheter assembly 300, the docking site 116 (FIGS. 1A-1C) and nesting site 136 (FIGS. 1A-1C) of the catheter assembly 100 (FIGS. 1A-1C), and/or the docking site 216 (FIGS. 2A-2D) and nesting site 236 (FIGS. 2A-2D) of the catheter assembly 200 (FIGS. 2A-2D) may be interchanged in any of the above-mentioned embodiments. That is, for example, the working catheter 230 (FIGS. 2A-2D) and the delivery catheter 210 (FIGS. 2A-2D), in embodiments, could selectively couple by means of the threaded docking site 316 and nesting site 336 discussed with respect to FIGS. 3A-3D. Moreover, for example, it should be appreciated that the biasing rail 366 may readily be incorporated in the catheter assemblies 100 (FIGS. 1A-1C) and 200 (FIGS. 2A-2D).

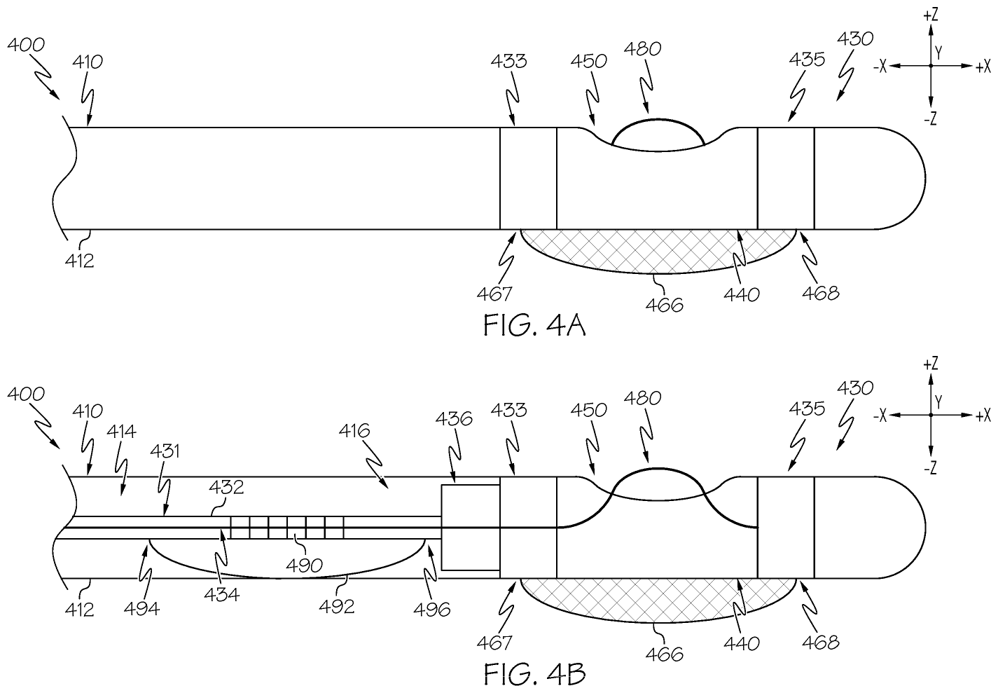

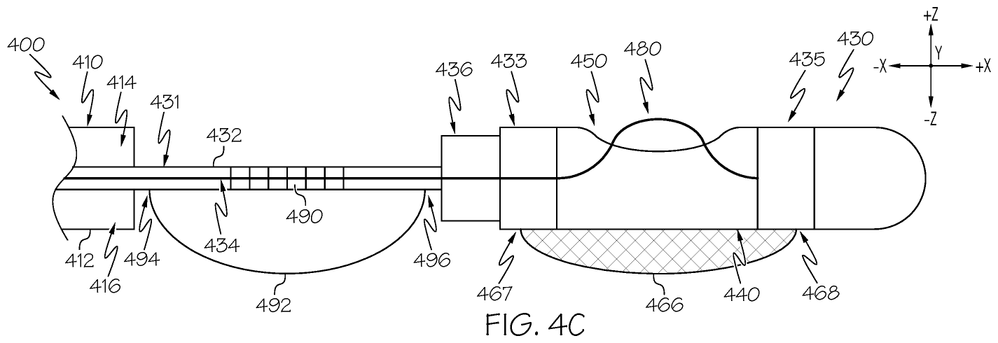

Referring now to FIGS. 4A-4C a catheter assembly 400 is depicted. The catheter assembly 400 may resemble the catheter assembly 100 discussed in FIGS. 1A-1D, the catheter assembly 200 discussed in FIGS. 2A-2D, and/or the catheter assembly 300 discussed in FIGS. 3A-3D in all aspects except as discussed herein. For instance, the catheter assembly 400 generally includes a delivery catheter 410 having a delivery catheter body 412 and one or more lumens 414 or other passageways extending at least partially along or through the delivery catheter body 412. The catheter assembly 400 further includes a working catheter 430 having a working catheter body 431. A first portion 432 of the working catheter body 431 is able to be received within the delivery catheter 410. The working catheter 430 may further include one or more lumens 434 or other passageways extending at least partially along or through the working catheter body 431. At least a portion of the working catheter 430 is more flexible than the delivery catheter 410. Particularly, the first portion 432 of the working catheter body 431 may be more flexible than the delivery catheter 410. The working catheter 430 further includes a working site 450 that allows for passage of one or more instruments, such as the electrode 480, into and/or out of the working catheter body 431. The working catheter 430 of the catheter assembly 400 further includes a nesting site 436, and the delivery catheter 410 further includes a docking site 416, which allow for selectively coupling the working catheter 430 with the delivery catheter 410. The working catheter 430 may include a second portion 433 and/or a third portion 435.

The working catheter 430 includes a self-expanding cage 466. The self-expanding cage 466 may resemble the biasing rail 266 (FIGS. 2A-2D) in function and may be made of similar materials. In the extended configuration, the self-expanding cage 466 may have a three-dimensional dome shape. For instance, the self-expanding cage 466 may be made of metal, plastic, polymer, metal coated in plastic, a composite of any of said materials, and the like. For instance, the self-expanding cage 466 may be nitinol, stainless steel, polyethylene terephthalate, polyether ether ketone, polytetrafluoroethylene, polyimide, and the like. The self-expanding cage 466 may be a material that exhibits shape memory. The self-expanding cage 466 may be coupled to the working catheter 430 along a non-active side 440 of the working catheter body 431. The self-expanding cage 466 may be coupled to the working catheter body at a proximal point 467 and a distal point 468 such that the self-expanding cage 466 spans the working section. In embodiments, the working catheter 430 may include the first array of magnets 260 (FIGS. 2A-2D), second array of magnets 262 (FIGS. 2A-2D), and/or third array of magnets 264 (FIGS. 2A-2D) in the second portion 433, third portion 435, and working site 450, respectively. In such embodiments, the self-expanding cage 466 may longitudinally span the first array of magnets 260 (FIGS. 2A-2D), second array of magnets 262 (FIGS. 2A-2D), and/or third array of magnets 264 (FIGS. 2A-2D).

The working catheter 430 includes an array of magnets 490 positioned along the first portion 432 of the working catheter body 431. The array of magnets 490 may resemble the arrays of magnets 260, 262 (FIGS. 2A-2D) in form and function. That is, the array of magnets 490 may be fixed or adhered to the first portion 432 of the working catheter body 431 in similar fashion as the first array of magnets 260 (FIGS. 2A-2D) to the second portion 233 (FIGS. 2A-2D) of the working catheter body 231 (FIGS. 2A-2D). In embodiments, the outer surface of the array of magnets 490 may be flush or in line with the outer surface of the first portion 432 of the working catheter body 431. Particularly, the array of magnets 490 are shaped and sized such that the array of magnets 490 and the first portion 432 of the working catheter body 431 may be advanced out of the delivery catheter 410. That is, when the working catheter 430 and the delivery catheter 410 are coupled, the array of magnets 490 may be positioned within the lumen 414 of the delivery catheter 410. When the working catheter 430 and the delivery catheter 410 are decoupled, the working catheter 430 may be advanced such that at least a segment of the first portion 432 of the working catheter body 431 and the array of magnets 490 may be advanced out of the delivery catheter 410.

The working catheter 430 may include a biasing rail 492. The biasing rail may resemble the biasing rail 266 (FIGS. 2A-2D) in structure and function. For instance, the biasing rail 492 may be made of metal, plastic, polymer, metal coated in plastic, a composite of any of said materials, and the like. For instance, the biasing rail 492 may be nitinol, stainless steel, polyethylene terephthalate, polyether ether ketone, polytetrafluoroethylene, polyimide, and the like. The biasing rail 492 may be a material that exhibits shape memory. The biasing rail may be coupled to the working catheter 430 along the non-active side 440 of the working catheter body 431. The biasing rail 492 may be coupled to the working catheter body 431 at a proximal point 494 and a distal point 496. The proximal point 494 and the distal point 496 may be positioned along the first portion 432 of the working catheter body 431. In embodiments, the biasing rail 492 may span the array of magnets 490. Similar to the biasing rail 266 (FIGS. 2A-2D), the biasing rail 492 may be biased from a low-profile configuration to an extended configuration. For instance, when the working catheter 430 and the delivery catheter 410 are coupled, the biasing rail may be positioned within the lumen 414 of the delivery catheter body 412 such that the delivery catheter body 412 maintains the biasing rail 492 in the low-profile configuration. When the working catheter 430 is decoupled from the delivery catheter 410, at least a segment of the first portion 432 of the working catheter body 431 and the biasing rail 492 may be advanced from the delivery catheter 410 such that the delivery catheter no longer applies a force to the biasing rail 492 and the biasing rail 492 self-expands from the low-profile configuration to the extended configuration. The biasing rail 492 may further resemble the biasing rail 366 (FIGS. 3A-3D), in that the biasing rail 492 may extend into the lumen 434 of the working catheter 430 at an opening at the proximal point 494. In such embodiments, the biasing rail 492 may be user-manipulated to extend from the low-profile configuration to the extended configuration.

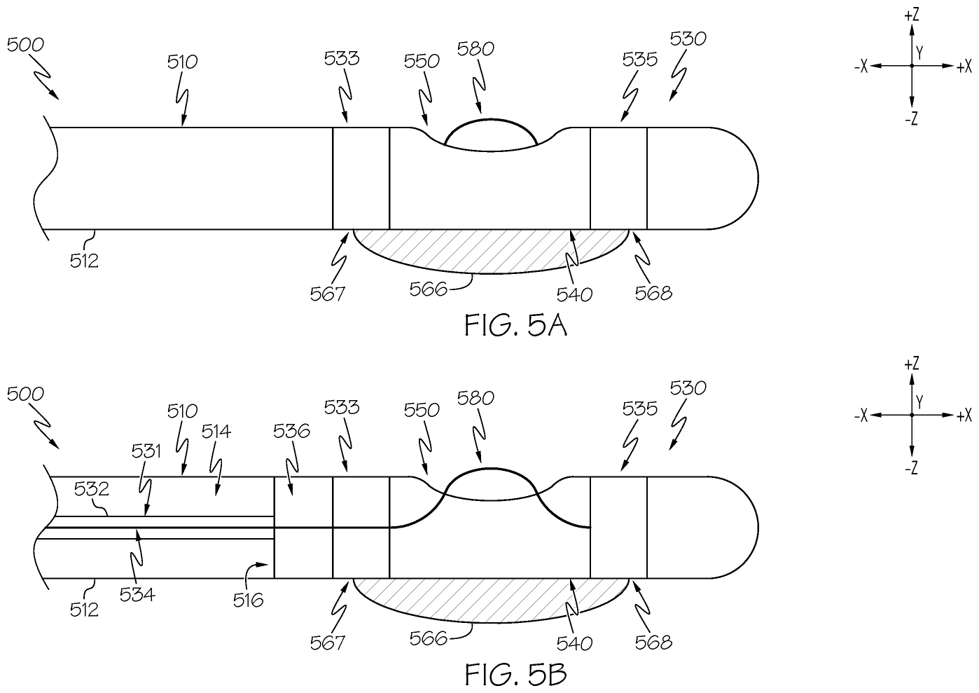

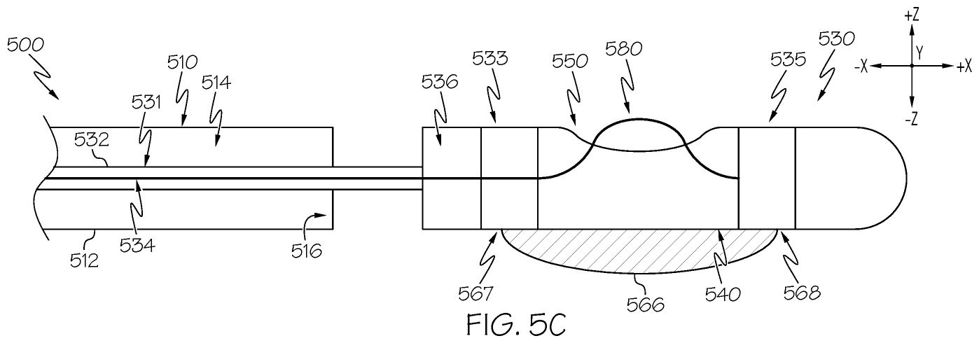

It should be appreciated that the working catheter 430 may include the array of magnets 490 and the biasing rail 492 independently of each other. Moreover, it should be appreciated that the biasing rail 492 may be replaced with a self-expanding cage, similar to the self-expanding cage 466, or a balloon 566 (discussed with reference to FIGS. 5A-5C). The array of magnets 490 and/or the biasing rail 492 may function to bias the first portion 432 of the working catheter body 431 toward a target vessel wall when the working catheter 430 is decoupled from the delivery catheter 410. Therefore, the array of magnets 490 and/or the biasing rail 492 may prevent the first portion 432 of the working catheter body 431, having a high flexibility, from “swimming,” or uncontrollably shifting, in a blood vessel.

It should be appreciated that the docking site 416 and nesting site 436 of the catheter assembly 400 may take the form and function of any of the docking sites 116 (FIGS. 1A-1C), 216 (FIGS. 2A-2D), 316 (FIGS. 3A-3D) and nesting sites 136 (FIGS. 1A-1C), 236 (FIGS. 2A-2D), 336 (FIGS. 3A-3D) previously discussed. Similarly, the self-expanding cage 466 may be readily interchanged with the biasing rail 266 (FIGS. 2A-2D) and/or the biasing rail 366 (FIGS. 3A-3D) and vice versa. Moreover, the array of magnets 490, biasing rail 492, and self-expanding cage 466 may be incorporated in any of the working catheters 130 (FIGS. 1A-1C), 230 (FIGS. 2A-2D), 330 (FIGS. 3A-3D) previously discussed.

Referring now to FIGS. 5A-5C a catheter assembly 500 is depicted. The catheter assembly 500 may resemble the catheter assembly 100 discussed in FIGS. 1A-1D, the catheter assembly 200 discussed in FIGS. 2A-2D, the catheter assembly 300 discussed in FIGS. 3A-3D, and/or the catheter assembly 400 discussed in FIGS. 4A-4C in all aspects except as discussed herein. For instance, the catheter assembly 500 generally includes a delivery catheter 510 having a delivery catheter body 512 and one or more lumens 514 or other passageways extending at least partially along or through the delivery catheter body 512. The catheter assembly 500 further includes a working catheter 530 having a working catheter body 531. A first portion 532 of the working catheter body 531 is able to be received within the delivery catheter 510. The working catheter 530 may further include one or more lumens 534 or other passageways extending at least partially along or through the working catheter body 531. At least a portion of the working catheter 530 is more flexible than the delivery catheter 510. Particularly, the first portion 532 of the working catheter body 531 may be more flexible than the delivery catheter 510. The working catheter 530 further includes a working site 550 that allows for passage of one or more instruments, such as the electrode 580, into and/or out of the working catheter body 531. The working catheter 530 of the catheter assembly 500 further includes a nesting site 536, and the delivery catheter 510 further includes a docking site 516, which allow for selectively coupling the working catheter 530 with the delivery catheter 510. The working catheter 530 may include a second portion 533 and/or a third portion 535.

The working catheter 530 includes a balloon 566. The balloon 566 may be controllably expanded (e.g., controllably filled with saline or other fluid). The balloon 566 may be an asymmetrical balloon. The balloon 566 may be coupled to the working catheter 530 along a non-active side 540 of the working catheter body 531. The balloon 566 may be coupled to the working catheter body 531 at a proximal point 567 and a distal point 568 such that the balloon 566 spans the working site 550. In embodiments, the working catheter 530 may include the first array of magnets 260 (FIGS. 2A-2D), second array of magnets 262 (FIGS. 2A-2D), and/or third array of magnets 264 (FIGS. 2A-2D) in the second portion 533, third portion 535, and working site 550, respectively. In such embodiments, the balloon 566 may longitudinally span the first array of magnets 260 (FIGS. 2A-2D), second array of magnets 262 (FIGS. 2A-2D), and/or third array of magnets 264 (FIGS. 2A-2D). Similar to the biasing rail 266 (FIGS. 2A-2D), biasing rail 366 (FIGS. 3A-3D), self-expanding cage 466, the balloon 566 may bias the working site 550 and electrode 580 against a target vessel wall.

It should be appreciated that the docking site 516 and nesting site 536 of the catheter assembly 500 may take the form and function of any of the docking sites 116 (FIGS. 1A-1C), 216 (FIGS. 2A-2D), 316 (FIGS. 3A-3D) and nesting sites 136 (FIGS. 1A-1C), 236 (FIGS. 2A-2D), 336 (FIGS. 3A-3D) previously discussed. Similarly, the balloon 566 may be readily interchanged with the biasing rail 266 (FIGS. 2A-2D), the biasing rail 366 (FIGS. 3A-3D), and/or the self-expanding cage 466 (FIGS. 4A-4C) and vice versa. Moreover, the array of magnets 490 (FIGS. 4A-4C) and biasing rail 492 (FIGS. 4A-4C) may be incorporated in the working catheter 530.

While biasing rails, self-expanding cages, and balloons have been discussed in detail herein, it should be appreciated that the catheter assemblies discussed herein may include any suitable biasing element configured to extend radially outward from the non-active side in order to bias the electrode or modification device, and the disclosure is not limited to rails, cages, and/or balloons.

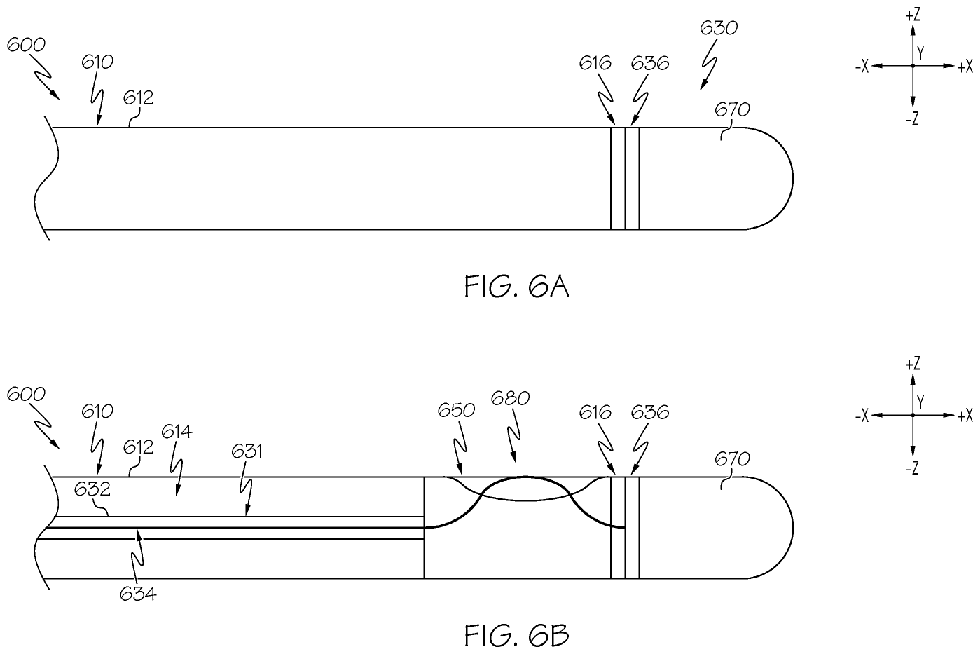

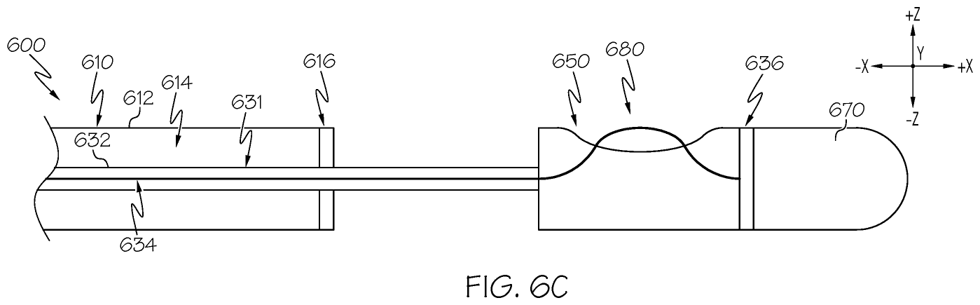

Referring now to FIGS. 6A-6C, a catheter assembly 600 is depicted. The catheter assembly 600 may resemble the catheter assembly 100 discussed in FIGS. 1A-1D, the catheter assembly 200 discussed in FIGS. 2A-2D, the catheter assembly 300 discussed in FIGS. 3A-3D, the catheter assembly 400 discussed in FIGS. 4A-4C, and/or the catheter assembly 500 discussed in FIGS. 5A-5C in all aspects except as discussed herein. For instance, the catheter assembly 600 generally includes a delivery catheter 610 having a delivery catheter body 612 and one or more lumens 614 or other passageways extending at least partially along or through the delivery catheter body 612. The catheter assembly 600 further includes a working catheter 630 having a working catheter body 631. A first portion 632 of the working catheter body 631 is able to be received within the delivery catheter 610. The working catheter 630 may further include one or more lumens 634 or other passageways extending at least partially along or through the working catheter body 631. At least a portion of the working catheter 630 is more flexible than the delivery catheter 610. Particularly, the first portion 632 of the working catheter body 631 may be more flexible than the delivery catheter 610. The working catheter 630 further includes a working site 650 that allows for passage of one or more instruments, such as the electrode 680, into and/or out of the working catheter body 631. The working catheter 630 of the catheter assembly 600 further includes a nesting site 636, and the delivery catheter 610 further includes a docking site 616, which allow for selectively coupling the working catheter 630 with the delivery catheter 610.

The working site 650 is sized relative to the delivery catheter body 612 and lumen 614, and positioned relative to the nesting site 636 such that the working site 650 and the electrode 680 are housed within the delivery catheter 610 when the delivery catheter 610 and working catheter 630 are coupled. That is, the working site 650 may have a smaller diameter than the delivery catheter body 612 such that the working site 650 may be received within the lumen 614. The nesting site 636 may be positioned distal (e.g. in the +x direction of the coordinate axes of FIGS. 6A-6C) the working site 650. The nesting site 636 may be positioned distal the working site 650 and proximal (e.g. in the −x direction of the coordinate axes of FIGS. 6A-6C) a distal tip 670 of the working catheter 630. In embodiments, the working catheter 630 may include the second portion 233 (FIGS. 2A-2D) distal the first portion 632 and proximal the working site 650. In such embodiments, the second portion 233 (FIGS. 2A-2D) may also have a smaller diameter than the delivery catheter body 612 such that the second portion 233 (FIGS. 2A-2D) may be received within the lumen 614 and is housed within the delivery catheter 610 when the delivery catheter 610 and the working catheter 630 are coupled. In embodiments, the working catheter 630 may include the third portion 235 (FIGS. 2A-2D) distal the working site 650 and proximal the distal tip 670. The third portion 235 (FIGS. 2A-2D) may be proximal the nesting site 636. In such embodiments, the third portion 235 (FIGS. 2A-2D) may also have a smaller diameter than the delivery catheter body 612 such that the third portion 235 (FIGS. 2A-2D) may be received within the lumen 614 and is housed within the delivery catheter 610 when the delivery catheter 610 and the working catheter 630 are coupled. In embodiments, the third portion 235 (FIGS. 2A-2D) may be distal the nesting site 636 and proximal the distal tip 670. In such embodiments, the third portion 235 (FIGS. 2A-2D) may be exposed from the delivery catheter 610 when the delivery catheter 610 and working catheter 630 are coupled.

It should be appreciated that the docking site 616 and nesting site 636 of the catheter assembly 600 may take the form and function of any of the docking sites 116 (FIGS. 1A-1C), 216 (FIGS. 2A-2D), 316 (FIGS. 3A-3D) and nesting sites 136 (FIGS. 1A-1C), 236 (FIGS. 2A-2D), 336 (FIGS. 3A-3D) previously discussed. For instance, the docking site 616 may include a docking magnet 118 (FIGS. 1A-1C) and the nesting site 636 may include a nesting magnet 138 (FIGS. 1A-1C). In other embodiments, the nesting site 636 may be sized to be received in the docking site 616 such that nesting site 636 and docking site 616 are friction fit. In yet other embodiments, the docking site 616 may include docking threads 318 (FIGS. 3A-3D) and the nesting site 636 may include nesting threads 338 (FIGS. 3A-3D).

It should further be appreciated that the working catheter 630 may include any of the biasing mechanisms discussed in previous embodiments. For instance, the working catheter 630 may include any or all of the first array of magnets 260 (FIGS. 2A-2D), second array of magnets 262 (FIGS. 2A-2D), third array of magnets 264 (FIGS. 2A-2D), biasing rail 266 (FIGS. 2A-2D), biasing rail 366 (FIGS. 3A-3D), self-expanding cage 466 (FIGS. 4A-4C), and balloon 566 (FIGS. 5A-5C). In such embodiments, the delivery catheter 610 may be sized such that the biasing mechanism, such as the biasing rail 266 (FIGS. 2A-2D) may be received within the delivery catheter 610 and maintained in a low-profile configuration when the working catheter 630 and the delivery catheter 610 are coupled. Moreover, the array of magnets 490 (FIGS. 4A-4C) and biasing rail 492 (FIGS. 4A-4C) may be incorporated in the working catheter 630.

Referring now to FIGS. 7A and 7B a system and method for forming a fistula between the first blood vessel 700 and a second blood vessel 702 with the catheter assembly 200 (e.g. the first catheter) and the second catheter 900 will now be discussed. Referring first to FIG. 7A, the catheter assembly 200, with the working catheter 230 and the delivery catheter 210 coupled, may be advanced within a lumen of the blood vessel 700. The second catheter 900 may be placed in a blood vessel 702 that is adjacent to the blood vessel 700. The second catheter 900 may resemble the catheter assemblies previously discussed herein. That is, the second catheter 900 may be a catheter assembly having a delivery catheter and working catheter that are selectively couplable. In some embodiments, the second catheter 900 may include an electrode or other fistula forming device, similar to the catheter assembly 200. In some embodiments, the second catheter 900 may not include an electrode or other fistula forming device. The second catheter 900 includes a catheter body 910. The catheter body 910 further defines a working site 950 having an active side 952. The second catheter 900 further includes a non-active side defined by the catheter body 910 and the working site 950. Specifically, the active side 952 of the working site 950 may include a recess 980 configured to receive the electrode 280 of the catheter assembly 200. The recess 980 may be particularly shaped, sized, and/or the like to receive the electrode 280 of the catheter assembly 200 therein. In other embodiments, the second catheter 900 may include an electrode that extends from the working site 950 and radially away from the catheter body 910. The catheter body 910 of the second catheter 900 may further include one or more arrays of magnets. For instance, the second catheter 900 may include a first array of magnets 960 positioned proximal (e.g. in the +x direction of the coordinate axes of FIGS. 7A and 7B) to the working site 950 and a second array of magnets 962 positioned distal (e.g. in the −x direction of the coordinate axes of FIGS. 7A and 7B) to the working site 950. In embodiments, the catheter 900 may include a third array of magnets positioned along the working site 950, and particularly along the non-active side of the working site 950. In embodiments, the second catheter 900 may include any or all of the biasing mechanisms previously discussed. For instance, the second catheter 900 may include a biasing rail, a self-expanding cage, and/or a balloon to bias the active side 952 of the working site 950 against a target vessel wall.

The first and second arrays of magnets 260, 262 of the catheter assembly 200 and the first and second arrays of magnets 960, 962 of the second catheter 900 may be configured to promote rotational and axial alignment of the catheter assembly 200, and particularly the working catheter 230, and the second catheter 900. Proper axial and rotational alignment between the catheter assembly 200 and second catheter 900 may facilitate alignment of one or more fistula-forming elements, such as the working sites 150, 950 of the catheter assembly 200 and second catheters 900, respectively. More specifically, proper axial and rotational alignment between the catheter assembly 200 and the second catheter 900 may facilitate alignment of the electrode 280 with the recess 980. The one or more arrays of magnets 260, 262 of the catheter assembly 200 may be arranged such that the magnetic fields generated by the one or more arrays of magnets 260, 262 are stronger in the direction of the active side 252 of the working site 250 (e.g. in the −z direction of the coordinate axes of FIGS. 7A and 7B) than in the direction of the non-active side 240 of the working site 250 (e.g. in the +z direction of the coordinate axes of FIGS. 7A and 7B). Similarly, the one or more arrays of magnets 960, 962 of the second catheter 900 may be arranged such that the magnetic fields generated by the one or more arrays of magnets 960, 962 are stronger in the direction of the active side 952 of the working site 950 (e.g. in the +z direction of the coordinate axes of FIGS. 7A and 7B) than in the direction of the non-active side of the working site 950 (e.g. in the −z direction of the coordinate axes of FIGS. 7A and 7B). In such embodiments, the strength of the magnetic fields in the directions of the active sides 252, 952 of the working sites 250, 950, respectively, may promote rotational alignment between the active side 252 of the working site 250 of the catheter assembly 200 in the first blood vessel 700 and the active side 952 of the working site 950 of the second catheter 900 in the second blood vessel 702.

The catheter assembly 200 and second catheter 900, as depicted in FIG. 7A are axially misaligned, such that the electrode 280 of the catheter assembly 200 is not aligned with the recess 980 of the second catheter 900 in the x-direction of the coordinate axes of FIGS. 7A and 7B. Moreover, as depicted in FIG. 7A, for instance, the catheter assembly 200 and the second catheter 900 are in weak coaptation. When in weak coaptation, space may remain between at least one of the active side 252 of the working site 250 of the catheter assembly 200 and an adjacent wall of the blood vessel 700 and the active side 952 of the working site 950 of the second catheter 900 and an adjacent wall of the blood vessel 702. Therefore, in weak coaptation, the active side 252 of the working site 250 and the active side 952 of the working site 950 are not in close approximation with one another (e.g. in the direction of the z-axis of the coordinate axes of FIGS. 7A and 7B).

Each array of magnets 260, 262 of the catheter assembly 200 may be configured to mate with a corresponding array of magnets 960, 962 of the second catheter 900, and vice versa, such that the catheter assembly 200 and the second catheter 900 may be aligned and coapted. As used herein, the terms “coapted” and/or “strong coaptation” may be understood to mean that the catheter assembly 200, and particularly the working catheter 230, and the second catheter 900 are in close approximation (e.g. in the direction of the z-axis of the coordinate axes of FIGS. 7A and 7B) such that the electrode 280 of the catheter assembly 200 may enter the recess 980 of the second catheter 900. The catheter assembly 200 may further include the biasing rail 266 to further promote alignment and strong coaptation between the catheter assembly 200 and the second catheter 900. For instance, in the extended configuration, the biasing rail 266 may apply a biasing force against the wall of the blood vessel 700, resulting in a biasing reaction force to be applied to the catheter assembly 200 to push the active side 252 and/or the one or more arrays of magnets 260, 262 against a wall of the blood vessel 700. It should be appreciated that the self-expanding cage 466 (FIGS. 4A-4C) and the balloon 566 (FIGS. 5A-5C) apply a similar biasing reaction force as the biasing rail 266.

During a fistula-forming procedure, however, the one or more arrays of magnets 260, 262 of the catheter assembly 200, the one or more arrays of magnets 960, 962 of the second catheter 900, and the biasing rail 266 may be insufficient to align and coapt the working site 250 and the working site 950 when the working catheter 230 and delivery catheter 210 of the catheter assembly 200 are coupled. For instance, due to the limited flexibility of the delivery catheter 210 and the tortuous anatomy of the first blood vessel 700, the arrays of magnets 260, 262 and biasing rail 266 may be unable to bias the working site 250 against a target wall of the vessel 700. Moreover, and the arrays of magnets 260, 262 may be unable to mate with the arrays of magnets 960, 962 of the second catheter 900. Therefore, as shown in FIG. 7A the first catheter assembly 200 and the second catheter 900, and more particularly the working site 250 and the working site 950, may be axially misaligned and/or in weak cooptation.

To promote alignment and coaptation of the working sites 250 with the working site 950, a user may decouple the working catheter 230 from the delivery catheter 210 of the catheter assembly 200, as discussed above. In other words, the working catheter 230, including the working site 250, and at least a portion of which has a greater flexibility than the delivery catheter 210, may be freed from the stiff and inflexible delivery catheter 210. Therefore, by not being limited by the stiffness of the delivery catheter 210, the working site 250 of the working catheter 230 can be fully aligned and coapted with the working site 950.

More particularly, in embodiments, a user may advance the catheter assembly 200, with the working catheter 230 and the delivery catheter 210 coupled, distally in the blood vessel 700. The user may manipulate the coupled catheter assembly 200 such that the working site 250 is generally longitudinally aligned with a target site to form a fistula. The user may also manipulate the coupled catheter assembly 200 such that the working site 250 is generally rotationally aligned with the target site to form a fistula with the electrode 280, for example. That is, a user may rotate the catheter assembly 200 such that the working site 250 is substantially facing the target site to form a fistula. However, as explained above, the stiffness of the delivery catheter 210 may prevent full coaptation and alignment of the working site 250 with the fistula site when the working catheter 230 is coupled with the delivery catheter 210. Therefore, the user may decouple the working catheter 230 and the delivery catheter 210, such that the flexibility of the working catheter 230 is not limited by the flexibility of the delivery catheter 210, and withdraw the delivery catheter 210 a distance proximally in the blood vessel 700. Once the working catheter 230 is decoupled, the one or more array of magnets 260, 262 and/or the biasing rail 266 may be sufficient to fully coapt and align the working site 250 with the fistula site and/or the working site 950. That is, the user may not need to individually manipulate the working catheter 230 to fully align and coapt the working site 250.

In some embodiments, however, the user may not closely longitudinally and rotationally align the working site 250 with the fistula site when the working catheter 230 is coupled with the delivery catheter 210. In such embodiments, after decoupling the working catheter 230 from the delivery catheter 210, the one or more arrays of magnets 260, 262 and the biasing rail 266 may not be able to fully align and coapt the working site 250 with the fistula site without additional user manipulation. Therefore, by means of a handle at the proximal end of the working catheter 230, for instance, the user may independently manipulate the working catheter 230 in the blood vessel 700 after decoupling. In doing so, the user may longitudinally position the working site 250 in the blood vessel 700 with sufficient accuracy such that the biasing rail, and the interaction between the magnets 260, 262 and the magnets 960, 962 can generate full alignment and coaptation between the working site 250 and the fistula site and/or working site 950. Similarly, instead of manipulating the working catheter 230 at its proximal end, a user may steer or manipulate the positioning of the working catheter 230 in the blood vessel 700 by means of one or more external magnets.

Once the fistula is formed between the blood vessels 700 and 702, as noted above, the user may manipulate the working catheter 230 and/or the delivery catheter 210 to re-couple the working catheter 230 and delivery catheter 210 at the nesting site 236 and docking site 216. Therefore, the catheter assembly 200, with the working catheter 230 and delivery catheter 210 coupled, may be withdrawn from the blood vessel 700.

Embodiments can be described with reference to the following numerical clause:

1. A catheter assembly, comprising: a delivery catheter comprising a delivery catheter body defining an inner lumen of the delivery catheter; and a working catheter comprising a modification device, wherein: the working catheter extends through the inner lumen of the delivery catheter; and the working catheter is selectively couplable with the delivery catheter.

2. The catheter assembly of clause 1, wherein at least a portion of the working catheter is more flexible than the delivery catheter.

3. The catheter assembly of any preceding clause, wherein advancement of the delivery catheter through a blood vessel simultaneously advances the working catheter through the blood vessel when the working catheter is coupled with the delivery catheter.

4. The catheter assembly of any preceding clause, wherein the working catheter is advanceable through a blood vessel separately from the delivery catheter when the working catheter is decoupled from the delivery catheter.

5. The catheter assembly of any preceding clause, wherein the working catheter further comprises a nesting site, wherein the nesting site is configured to selectively couple with the delivery catheter.

6. The catheter assembly of any preceding clause, wherein the nesting site is proximal to the modification device.

7. The catheter assembly of any preceding clause, wherein the delivery catheter further comprises a docking site, wherein the working catheter is configured to selectively couple with the delivery catheter at the docking site.

8. The catheter assembly of any preceding clause, wherein the docking site is at a distal end of the delivery catheter.

9. The catheter assembly of any preceding clause, wherein: the working catheter further comprises a nesting site including a nesting magnet; and the delivery catheter further comprises a docking site including a docking magnet, wherein: the nesting magnet is configured to selectively magnetically couple with the docking magnet to couple the working catheter to the delivery catheter.

10. The catheter assembly of any preceding clause, wherein: the working catheter further comprises a nesting site; and the delivery catheter further comprises a docking site, wherein: the nesting site is configured to form a friction fit with the docking site to couple the working catheter to the delivery catheter.

11. The catheter assembly of any preceding clause, wherein: the working catheter further comprises a nesting site including nesting threads; and the delivery catheter further comprises a docking site including docking threads, wherein: the nesting site is configured to selectively threadedly couple with the docking site to couple the working catheter to the delivery catheter.

12. The catheter assembly of any preceding clause, wherein the modification device is positioned within the inner lumen of the delivery catheter when the working catheter is coupled with the delivery catheter.

13. The catheter assembly of any preceding clause, wherein the working catheter further comprises a working site, wherein the modification device is configured to radially extend away from the working catheter at the working site.

14. The catheter assembly of any preceding clause, wherein the working site comprises a heat-insulating material.

15. The catheter assembly of any preceding clause, wherein the working catheter further comprises one or more magnets.

16. The catheter assembly of any preceding clause, wherein the working catheter further comprises: a working site, wherein the modification device is configured to radially extend away from the working catheter at the working site; and one or more arrays of magnets longitudinally positioned along the working catheter relative to the working site.

17. The catheter assembly of any preceding clause, wherein the working catheter further comprises: a working site, wherein the modification device is configured to radially extend away from the working catheter at the working site; and the one or more magnets are positioned within the working site of the working catheter.

18. The catheter assembly of any preceding clause, wherein the working catheter further comprises one or more biasing rails, wherein: the one or more biasing rails longitudinally extend along a length of the working catheter and are configured to radially arch away from a non-active side of the working catheter such that the one or more biasing rails are configured to bias the modification device against a first blood vessel wall.

19. The catheter assembly of any preceding clause, wherein the working catheter further comprises a balloon configured to bias the modification device against a first blood vessel wall.

20. The catheter assembly of any preceding clause, wherein the working catheter further comprises an expandable cage configured to bias the modification device against a first blood vessel wall.

21. The catheter assembly of any preceding clause, wherein the modification device is an electrode, and the working catheter further comprises a lead wire electrically coupled to the electrode and positioned within a working catheter body, wherein the working catheter body comprises an insulative material.

22. The catheter assembly of any preceding clause, wherein the working catheter body comprises polytetrafluoroethylene or polyether block amide.

23. A method of forming a fistula between a first blood vessel and a second blood vessel comprising: advancing a first catheter assembly into the first blood vessel, wherein the first catheter assembly comprises: a delivery catheter comprising a delivery catheter body defining an inner lumen of the delivery catheter; and a working catheter comprising a modification device, wherein: the working catheter extends through the inner lumen of the delivery catheter; and the working catheter is selectively coupled with the delivery catheter; decoupling the working catheter from the delivery catheter such that the working catheter and delivery catheter are independently moveable relative to each other; and modifying tissue with the modification device of the working catheter.

24. The method of clause 23, further comprising: advancing the first catheter assembly to a first point in the first blood vessel when the working catheter is coupled to the delivery catheter; and advancing the working catheter to a second point in the first blood vessel after the working catheter is decoupled from the delivery catheter.

25. The method of any preceding clause, wherein the tissue is modified with the modification device of the working catheter after the working catheter is decoupled from the delivery catheter.

26. The method of any preceding clause, further comprising advancing a second catheter into the second blood vessel.

27. The method of any preceding clause, further comprising aligning the first catheter assembly and the second catheter at a site to form the fistula.

28. The method of any preceding clause, further comprising coapting the first catheter assembly and the second catheter at a site to form the fistula.

29. The method of any preceding clause, wherein coapting the first catheter assembly and the second catheter further comprises coapting the working catheter with the second catheter after the working catheter is decoupled from the first catheter assembly.

30. The method of any preceding clause, further comprising: advancing the first catheter assembly to a first point in the first blood vessel when the working catheter is coupled to the delivery catheter; and retracting the delivery catheter to a second point in the first blood vessel after the working catheter is decoupled from the delivery catheter.

31. A system for forming a fistula between two blood vessels, comprising: a first catheter assembly comprising: a delivery catheter comprising a delivery catheter body defining an inner lumen of the delivery catheter; and a working catheter comprising a modification device, wherein: the working catheter extends through the inner lumen of the delivery catheter; and the working catheter is selectively couplable with the delivery catheter; and a second catheter.

32. The system of clause 31, wherein the first catheter assembly is configured to be positioned within a first blood vessel and the second catheter is configured to be positioned within a second blood vessel adjacent to the first blood vessel.

33. The system of any preceding clause, wherein the second catheter further comprises a recessed region defining an active site of the second catheter, the recessed region configured to receive the modification device of the working catheter.

34. The system of any preceding clause, wherein the second catheter comprises a second catheter body and one or more magnets arranged along the second catheter body.

35. The system of any preceding clause, wherein at least a portion of the working catheter is more flexible than the delivery catheter.

36. The system of any preceding clause, wherein advancement of the delivery catheter through a blood vessel simultaneously advances the working catheter through the blood vessel when the working catheter is coupled with the delivery catheter.

37. The system of any preceding clause, wherein the working catheter is advanceable through a blood vessel separately from the delivery catheter when the working catheter is decoupled from the delivery catheter.

It should now be understood that embodiments of the present disclosure are directed to devices, systems, and methods for forming a fistula between two blood vessels. In particular, the devices and methods for forming a fistula described herein may include a catheter assembly having a delivery catheter and a working catheter that may be selectively coupled. The delivery catheter includes a delivery catheter body and an inner lumen. The working catheter includes a working catheter body and a modification device, such as an electrode. The modification device may be configured to project from a working site of the working catheter and define an active side of the working catheter. The working catheter extends through the inner lumen of the delivery catheter. When coupled, the working catheter and the delivery catheter may be simultaneously manipulated in a blood vessel. When decoupled, the working catheter and the delivery catheter may be independently manipulated in a blood vessel. At least a portion of the working catheter is more flexible than the delivery catheter. More particularly, the limited flexibility of the delivery catheter may inhibit the ability to align and coapt the working site at a site to form a fistula when the working catheter and delivery catheter are coupled. When the working catheter and the delivery catheter are decoupled, the increased flexibility of the working catheter may promote alignment and coaptation of the working site with a site to form a fistula.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter assembly, comprising:
a delivery catheter comprising a delivery catheter body defining an inner lumen of the delivery catheter; and
a working catheter comprising a modification device wherein:
the working catheter extends through the inner lumen of the delivery catheter; and
the working catheter is selectively couplable with the delivery catheter.

2. The catheter assembly of claim 1, wherein at least a portion of the working catheter is more flexible than the delivery catheter.

3. (canceled)

4. The catheter assembly of claim 1, wherein the working catheter is advanceable through a blood vessel separately from the delivery catheter when the working catheter is decoupled from the delivery catheter.

5. The catheter assembly of claim 1, wherein the working catheter further comprises a nesting site, wherein the nesting site is configured to selectively couple with the delivery catheter.

6. (canceled)

7. The catheter assembly of claim 1, wherein the delivery catheter further comprises a docking site, wherein the working catheter is configured to selectively couple with the delivery catheter at the docking site.

8. (canceled)

9. The catheter assembly of claim 1, wherein:
the working catheter further comprises a nesting site including a nesting magnet; and
the delivery catheter further comprises a docking site including a docking magnet, wherein:
the nesting magnet is configured to selectively magnetically couple with the docking magnet to couple the working catheter to the delivery catheter.

10. The catheter assembly of claim 1, wherein:
the working catheter further comprises a nesting site; and
the delivery catheter further comprises a docking site, wherein:
the nesting site is configured to form a friction fit with the docking site to couple the working catheter to the delivery catheter.

11. The catheter assembly of claim 1, wherein:
the working catheter further comprises a nesting site including nesting threads; and
the delivery catheter further comprises a docking site including docking threads, wherein:
the nesting site is configured to selectively threadedly couple with the docking site to couple the working catheter to the delivery catheter.

12. The catheter assembly of claim 1, wherein the modification device is positioned within the inner lumen of the delivery catheter when the working catheter is coupled with the delivery catheter.

13. The catheter assembly of claim 1, wherein the working catheter further comprises a working site, wherein the modification device is configured to radially extend away from the working catheter at the working site.

14.-15. (canceled)

16. The catheter assembly of claim 1, wherein the working catheter further comprises:
a working site, wherein the modification device is configured to radially extend away from the working catheter at the working site; and
one or more arrays of magnets longitudinally positioned along the working catheter relative to the working site.

17. The catheter assembly of claim 1, wherein the working catheter further comprises:
a working site, wherein the modification device is configured to radially extend away from the working catheter at the working site; and
one or more magnets positioned within the working site of the working catheter.

18. The catheter assembly of claim 1, wherein the working catheter further comprises one or more biasing rails, wherein:
the one or more biasing rails longitudinally extend along a length of the working catheter and are configured to radially arch away from a non-active side of the working catheter such that the one or more biasing rails are configured to bias the modification device against a first blood vessel wall.

19. The catheter assembly of claim 1, wherein the working catheter further comprises a balloon configured to bias the modification device against a first blood vessel wall.

20. The catheter assembly of claim 1, wherein the working catheter further comprises an expandable cage configured to bias the modification device against a first blood vessel wall.

21. The catheter assembly of claim 1, wherein the modification device is an electrode, and the working catheter further comprises a lead wire electrically coupled to the electrode and positioned within a working catheter body, wherein the working catheter body comprises an insulative material.

22.-30. (canceled)

31. A system for forming a fistula between two blood vessels, comprising:
a first catheter assembly comprising:
a delivery catheter comprising a delivery catheter body defining an inner lumen of the delivery catheter; and
a working catheter comprising a modification device, wherein:
the working catheter extends through the inner lumen of the delivery catheter; and
the working catheter is selectively couplable with the delivery catheter; and
a second catheter.

32. The system of claim 31, wherein the first catheter assembly is configured to be positioned within a first blood vessel and the second catheter is configured to be positioned within a second blood vessel adjacent to the first blood vessel.

33. The system of claim 32, wherein the second catheter further comprises a recessed region defining an active site of the second catheter, the recessed region configured to receive the modification device of the working catheter.

34. The system of claim 32, wherein the second catheter comprises a second catheter body and one or more magnets arranged along the second catheter body.

35. The system of claim 31, wherein at least a portion of the working catheter is more flexible than the delivery catheter.

36.-37. (canceled)
